(19) United States
(12) Patent Application Publication
(10) Pub. No.: US 2024/0386997 A1
(43) Pub. Date: Nov. 21, 2024

(54) Random Epigenomic Sampling

(71) Applicant: XGenomes Corp., Cambridge, MA (US)

(72) Inventors: John Healy, Acton, MA (US); Kalim Mir, Cambridge, MA (US)

(73) Assignee: XGenomes Corp., Cambridge, MA (US)

(21) Appl. No.: 18/686,097

(22) PCT Filed: Aug. 25, 2022
(86) PCT No.: PCT/US2022/041594

§ 371 Date: Feb. 23, 2024

(51) Int. Cl. G16B 20/20; C12Q 1/6881; C12Q 1/6886; G16H 50/20

(52) U.S. Cl. CPC G16B 20/20; C12Q 1/6881; C12Q 1/6886; G16H 50/20; C12Q 2600/112; C12Q 2600/154

(57) ABSTRACT

Methods and systems for determining the presence of disease in a subject by determining the state of modification (e.g. methylation) of a random subset of loci across the genome by sequencing and/or methylation detection is provided, where the composition of the random subset may differ from one sample to another.

### CROSS REFERENCE TO RELATED APPLICATION

This claims priority to U.S. Provisional Patent Application No. 63/237,123 entitled “Random Epigenomic Sampling,” filed Aug. 25, 2021, which is hereby incorporated by reference.

### TECHNICAL FIELD

The present disclosure relates generally to systems and methods for determining whether a biological sample has a phenotype such as cancer by sites of epigenetic modification in genomic molecules from the biological sample.

### BACKGROUND

Phenotypes, trait and disease states are underscored by omics states comprising genome sequences, epigenetic states, transcriptomes etc. It would be biologically and clinically informative to obtain a molecular readout of the states of one or more omic types as a surrogate for phenotype/disease. This is particularly the case where the actual manifestation of the phenotype/disease is at its hidden or nascent stages or its re-emergence after treatment is not easy or possible to detect.

This is the case in cancer. The molecular mechanism that lead to symptomatic cancer are multi-step and are in play while the individual is asymptomatic. It would be of diagnostic and prognostic value to detect the omic states at these early stages. This could lead to individuals being routinely screened for cancer.

Abnormal DNA methylation is an early, frequent and persistent characteristic of cancer development (Aravanis et al., 2017). It has been shown that changes in DNA methylation status at loci across the genome can be detected in cell-free (cfDNA) in plasma. Nearly 30 million CpG sites across the human genome are modifiable from a methylated to unmethylated state or vice versa. By contrast each cancer genome only has a handful of cancer mutations. It is clear that changes in the state of methylation provides a signal for cancer far superior than obtained from mutations. Of note, it is clear from ENCODE (Encyclopedia of DNA elements) data that the patterns of DNA methylation have more in common between cancers than between tumor and healthy tissue of a particular cancer type (FIG. 1).

Abnormal hydroxymethylation has also been shown to be a characteristic of cancer genomes detectable in cfDNA (Bergamaschi et al 2020).

The detection of trace amounts of circulating DNA that can be identified as being derived from a tumor (ctDNA) can be utilized as a means to detect minimal residual disease, metastatic disease, cancer recurrence and, early detection, potentially in a pan-cancer manner. However, because the fraction of circulating cfDNA that is derived from tumor (the tumor fraction) is likely to be low (<0.01%) at early stages of cancer and after treatment, the detection of ctDNA is challenging.

ctDNA can be distinguished from other cfDNA by the detection of cancer mutations or the detection of changes in methylation states. The number of mutations in a cancer genome is 600 per genome and at low tumor fraction this is impossibly hard to detect—a typical blood draw will have very few to zero molecules that bear a cancer mutation. To boost the signal a large number of known tumor mutations can be monitored and a signal can be detected by machine learning (Zviran et al 2020).

Changes in methylation status can be monitored by bisulfite sequencing. However, the workflow is complex, damaging to DNA and yields noisy data with substantial false positives. Moreover, sequencing complete genomes and methylomes is costly (e.g. >$30,000) at the read depths required to detect tumor DNA in low tumor fraction, consequently both detection of mutations and changes in methylation (bisulfite sequencing) typically involves enrichment of specific sites in the genome. In many assays the sites to be enriched must be pre-selected by sequencing an actual tumor biopsy first. This is invasive for the patient and adds complexity to the laboratory workflow and may lead to loss of signal. However, complete sequencing at high depth is far too costly to be contemplated as a diagnostic or screening tool.

### SUMMARY

The present disclosure addresses the need in the art for devices, systems and methods for providing methods for detecting diseases such as cancer. In one broad aspect, the present disclosure is based on the counter-intuitive idea that the signal for presence of a disease such as cancer is detectable in a sample by random sampling of epigenetic status of sites across the genome, even when the tumor fraction is low.

The present disclosure is based on the counter-intuitive idea that the signal for presence of a disease such as cancer is detectable in a sample by random sampling of epigenetic status of sites across the genome, even when the tumor fraction is low. Some embodiments of the present disclosure make use of this random sampling directly on the genomic DNA without prior selection of loci, thus saving cost and time, and avoiding loss of sample material.

The disclosed systems and methods work on a random subset of molecules taken from a set of sample molecules, where the molecules that constitute the random subset may be different or only partially overlap from one sample to another. Moreover, sufficient sampling can be obtained from just a few genome equivalents and the signal for presence or absence of the phenotype or disease is more prominent where haplotypes of multiple epigenetically modifiable sites in the genome are considered. In some embodiments only CpGs that are hypomethylated in a large fraction of cancer patients but are hypomethylated in a fraction of healthy people constitute the epigenetic modification haplotype.

Thus, in some embodiments a method is provided for detecting a molecular signature comprising: (i) isolating a substantially random subset of molecules from a set of molecules in a nucleic acid sample, (ii) determining the identity of individual molecules within the subset of molecules by obtaining sequence information from each individual molecule using a sequencing or sequence detection method and using the sequence information to map the molecule in silico to a location in the genome, (iii) determining the epigenetic status of each of the molecules mapped to the genome in ii using a method for detecting presence or absence of, the extent of, or the pattern of methylation of individual molecules, (iv) aggregating data on the methylation status of individual molecules within the subset of molecules, and (v) determining a molecular signature based on the aggregated data.

In some such embodiments the composition of the substantially random subset is different from one sample to the next.

In some embodiments the epigenetic status or the state of modification comprises the state of 5-methylcytosine (5mC), 5-hydroxymethylcytosine (5hmC) or a combination thereof. Additional DNA modifications include 5-formylcytosine (5fC), 5-carboxylcytosine (5caC). In some embodiments the nucleic acid is RNA and one or more from the plethora of RNA modifications are determined. In addition some modifications are a result of DNA damage, for example oxidative DNA damage produces at least 20 modifications. In some embodiments the modification is on both Cs of a CpG dyad, in other embodiments the CpG dyad is hemi-methylated.

In some embodiments, the disclosed systems and methods determine the presence of disease or phenotype in a subject, by determining the state of modification of a substantially random subset of loci across the genome by a sequencing and/or methylation detection method, filtering the loci according to the extent to which they are methylated in populations with and without the disease, wherein the composition of the substantially random subset is different from one individual to another.

In some embodiments, the disclosed systems and methods detect a molecular signature for cancer by a method comprising: (i) isolating a substantially random subset of molecules from a set of molecules in a nucleic acid sample inside a device, (ii) determining the identity of individual molecules within the subset of molecules by obtaining sequence information from each individual molecule by using instrumentation for running a sequencing or sequence detection process inside the device and using the obtained sequence information to map the molecule in silico to a location in the genome using one or more computer processors and computer memory, (iii) determining the modification status of each of the molecules mapped to the genome in ii using a method for detecting presence or absence of, the extent of, or the pattern of modification of individual molecules, (iv) optionally executing a computer program to filter out, all the sites on individual molecules which do not fulfill a predefined criteria (e.g. have not been shown to correlate with the methylation status at that site on the same sequence in one or more cancer patients), (v) aggregating data on the methylation status of the individual molecules within the subset of molecules in computer memory (or remaining individual molecules within the subset of molecules in computer memory if step (iv) has been carried out), and (vi) using the proportion of molecules in the aggregated data whose methylation patterns has diverged from a baseline to obtain a molecular signature for the presence of cancer, as a computer output using predetermined thresholds.

In some embodiments the disclosed systems and methods comprises means for keeping the sample molecules well mixed and dispersed before they are isolated for analysis.

In some embodiments the nucleic acid sample (biological sample) comprises blood, plasma, urine, stool, saliva, sputum, throat swab, nasal swab, nasopharyngeal swab, ear swab, milk, hair follicle, skin, seroma or serosanguineous fluid, cerebrospinal fluid, or breath. In some embodiments the nucleic acid sample is a forensic sample, environmental sample.

In some embodiments the subset of molecules is substantially random, in that there has been no prior selection of molecular species. In some embodiments biases in various steps exist inadvertently, which prevent the sample from being completely random. In some embodiments, although there is no locus specific enrichment, the systems and method of the present disclosure allow for non-locus specific enrichment of modified sites using, for example, a methyl binding protein or anti-methyl C antibody to pull-down molecules containing methyl C. In some embodiments the randomness is after size selection of molecules. In some embodiments, the molecules are fragmented to within a specific size range, e.g. 30-60 nucleotides (nt) or 150-250 nt and are substantially random within this size range. Thus, in some embodiments the substantial randomness is with respect to not performing any locus specific enrichment. Locus-specific enrichment comprises physically selecting and collecting (typically using sequence-specific nucleic acid probes), cfDNA molecules containing previously determined parts of the genome, known for example to contain modifications which are known or suspected to be informative; this is done before the sequence or modification detection is done.

In some embodiments the nucleic acid samples are derived from plasma. In some embodiments DNA is analyzed. In some embodiments RNA as well as DNA, or as an alternative to DNA is analyzed.

In some embodiments proteins as well as, or as an alternative to nucleic acids are analyzed.

In some embodiments the size of DNA molecules is also analyzed. In some embodiments the fraction of cfDNA molecules in plasma that are around +/−10 nt from peak size of 167 nt are analyzed. In some embodiments the fraction of cfDNA molecules in plasma that are of other lengths are analyzed, for example there is a fraction of cfDNA that is typically around 10 Kb in length that may be included in analysis.

In some embodiments the extent of methylation/demethylation that is measured quantitatively by determining in an analog manner, the amount of signal corresponding to the number of methylated cytosines present. This is the case when a standard molecular probing or PCR methods are used.

In some embodiments the extent of methylation/demethylation is measured digitally by counting the number of occurrences of a base that has changed its methylation status from a reference (constituted from healthy samples) in the sequence reconstituted for an individual molecule in the sample using a next generation sequencing method. In some embodiments the extent of methylation is determined by a quantitative probing method. An example of the extent of hypomethylation (demethylation) of a particular molecule may be that the 160 nt length cell-free DNA (cfDNA) molecule has 7 CpG sites, and of those 7 sites 6 are methylated in one or more healthy samples used as a reference and, in the subject 5 methylated sites have become hypomethylated, so only 1 of the 7 sites remains methylated. This individual can be considered to show hypomethylation at this particular molecule. In some embodiments, to call hypomethylation for the purposes of constituting a molecular signature, these sites are further qualified. For example, only those sites out of the 7 that have previously been shown to be associated with cancer are taken into consideration. This constitutes one type of pre-defined criteria.

In some embodiments it is not only persistent hypomethylation in a string of CpG sites along a molecule that is looked for as a signal but strings of either hypo- or hyper-methylation sites are looked for. In some embodiments a string of switches of one methylation state to another along a single molecule, particularly when they have been seen before in cancer genomes, are taken as an indication that the molecule is derived from a tumor cell, providing evidence that a cancer phenotypes is present. In some embodiments the string of state switches is methylated to hypomethylated. In some embodiments the string of state switches is unmethylated to hypermethylated. In some embodiments the string of state switches are not homogeneously hypo- or hyper-methylation modifications but can be a mix of both as long as the state is switched from the state that is predominantly found in samples from healthy individuals.

In some embodiments, the extent of methylation is determined by looking at multiple sites along a molecule, and providing a qualitative or quantitative measurement without necessarily obtaining unequivocal evidence of which site is methylated or not methylated.

In some embodiments, the pattern of methylation is determined by looking at multiple sites along a molecule, and determining which site (e.g. CpG) along the individual molecule is methylated and which site is not. This then enables a haplotype for the molecule to be constructed. In some embodiments, the haplotype of individual molecules in a random subset of molecules is used to constitute the molecular signature.

In some such embodiments the disclosed systems and methods detect a molecular signature by a method comprising (i) isolating for analysis a substantially random subset of molecules from a set of molecules in a nucleic acid sample, (ii) determining the identity of individual molecules within the subset of molecules by obtaining sequence information from each individual molecule using a sequencing or sequence detection method and using the sequence information to map the molecule to a location in the genome, (iii) determining the methylation haplotype of each of the molecules mapped to the genome in ii using a method for detecting absence or presence of methylation along particular sequence sites (e.g. CpGs) along the molecule, and (iv) aggregating data on the methylation haplotype status of individual molecules within the subset of molecules to obtain a molecular signature. In some embodiments, (ii) and (iii) are obtained by the same process, e.g. bisulfite sequencing.

In some embodiments the signature is obtained by comparing the state of modification at sites in the test sample with a computer model of states per corresponding sites in the genome that correspond to specific sample disease or phenotype states.

Some such embodiments comprise a method for determining the presence or absence of, or the nature of, a particular disease or phenotype in a subject comprising: (i) determining the state of modification of a subset of modifiable sites across the genome to yield a matrix of state likelihoods per corresponding site in the genome, (ii) comparing the matrix of state likelihoods per corresponding site in the genome determined for the current sample against a computer model of states per corresponding site in the genome that correspond to specific sample disease or phenotype states, and (iii) determining the disease or phenotype state of the sample, as a whole, based on a threshold applied by the computer model. In some such embodiments, an individual site comprises multiple nucleotides in a contiguous part of the genome, represented on a single cell-free DNA molecule; this is the case where a site is a methylation haplotype block, which is a pattern of methylation across multiple CpG sites on a single DNA molecule derived from a single chromosome.

In some alternative embodiments, an individual site comprises multiple CpGs in non-contiguous parts of the genome, represented in cell-free DNA molecules in the sample. This is the case where two loci are functionally connected to each other, for example a modifier and its target gene (e.g. an enhancer or suppressor acting on a gene).

The common theme in such embodiments is that a relationship exists between more than one nucleotide.

In some embodiments such a relationship is already be known. In some embodiments, such a relationship is not be known before, or may not have been established through biological or genetic knowledge, but may be picked up by statistical methods such as principle components analysis or by machine learning.

In some embodiments, before the random subset of molecules is analyzed, a non-random selection is applied comprising enriching for CpGs.

In some embodiments, before the random subset of molecules is analyzed, a non-random deselection is applied comprising depleting Cot-1 (and in some cases Cot-2 fractions) of genomic DNA. In some embodiments, certain sequences are depleted from the set of molecules and a subset of this depleted set is used. In some embodiments the certain sequences are highly abundant sequences.

In some embodiments, the systems and method of the present disclosure provide a method for detecting a molecular signature for cancer comprising: (i) isolating for analysis a substantially random subset of molecules from a set of molecules in a nucleic acid sample, (ii) treating the isolated cell-free DNA molecules with bisulfite whereby unmethylated cytosines are converted to uracil, (iii) sequencing a random subset of bisulfite treated DNA molecules, (iv) aligning the sequence reads to a reference (e.g. a reference that takes into account bisulfite conversion) to determine the identity of the molecules, (v) building up the sequence and methylation status of the subset of molecules and optionally determining the extent and/or pattern of methylation by the bisulfite sequencing, (vi) aggregating data on the methylation status of individual molecules within the subset of molecules, and (vii) determining a molecular signature for cancer based on the aggregated data.

In some embodiments, an alternative to bisulfite treatment is used such as TET-assisted pyridine borane sequencing (TAPS; Exact Sciences, WI, USA), Enzymatic-methylation sequencing (EM-Seq/NEBNEXT; New England Biolabs, Ipswich, MA, USA).

In some embodiments the signature is obtained by comparing the state of modification at sites in the test sample with a computer model of states per corresponding sites in the genome that correspond to specific sample disease or phenotype states.

In some embodiments the isolating of step (i) in the embodiments above comprises dispersing and immobilizing the molecules on a surface in a manner that there is no pre-determined spatial organization of an individual molecule with respect to any other molecule on the surface. In some embodiments the arbitrary subset is defined by the area on the surface from where the data is collected. In some embodiments, an arbitrary subset of the molecules on the surface are analyzed, such subset in some embodiments being defined by the window of light illumination or light collection.

In some embodiments, the systems and method of the present disclosure provide a method for detecting a molecular signature for cancer comprising: (i) isolating cell-free DNA from plasma, (ii) sequencing a random subset of DNA molecules from the cell-free DNA using a sequencing method that can directly read methylation on the DNA (e.g., Pacific Biosciences, Oxford Nanopore Technologies, XGenomes sequencing technologies), (iii) aligning the sequence read to a reference, (iv) building up the sequence and methylation status of a subset of molecules and optionally the extent of methylation is measured by directly reading methylation on DNA, (v) aggregating data on the methylation status of individual molecules within the subset of molecules, and (vi) based on the aggregate data, obtaining a molecular signature

In some embodiments the isolating of step (i) comprises dispersing the molecules in a solution in a manner that there is no pre-determined spatial organization of an individual molecule with respect to any other molecule in a chamber comprising the sample. In some embodiments the subset is defined by molecules that enter a nanopore or a zero-mode waveguide within the time period of the analysis.

In some embodiments the molecular means for detection modification is repetitive transient binding of probes—short oligonucleotide or antibodies or modification-binding proteins—to the cell-free DNA (Mir, K. U.S. patent application Ser. Nos. 16/205,155 and 16/425,929).

In some such embodiments the method of detecting a signal for tumor DNA or cancer in a subject comprises: (i) obtaining a substantially random set of cell-free nucleic acid molecules from a subject, (ii) dispersing and fixing a substantially random subset of the random set of cell-free nucleic acid molecules on a surface, thus obtaining a random array of nucleic acid molecules within which array each molecule is fixed at a distinct location on the surface, (iii) exposing one or more probes (typically a repertoire or panel of oligos) of known identity to the nucleic acids, one or more of said probes capable of determining the identity of an individual nucleic acid molecule and detecting the binding of one or more of said probes to each individual nucleic acid in a subset of the dispersed molecules and determining the identity of the said each individual nucleic acid, (iv) exposing one or more probes of known identity to the nucleic acids, one or more of said probes capable of having a different binding profile when the sequence is modified compared to when the sequence is not modified and detecting the binding of one or more said probes to each individual nucleic acid in the same random subset as (iii) and determining if the binding profile better matches the binding profile of when the sequence is modified or the binding profile of when the sequence is not modified, (v) aggregating data from the subset of nucleic acid molecules in iii and iv and recording modification status of each of the molecules in the subset whose identity has been determined, and (vi) using the modification status of each identified molecule to determine a signal for the presence of tumor DNA and hence cancer.

In some embodiments the binding profile comprises whether binding has occurred or not. In some embodiments the binding profile is kinetic—the on time and off time of binding of fluorescently labeled probe is determined.

In some above embodiments the method comprises determining from the molecular signature whether cancer is present or not and if present its stage, its tissue of origin, its tissue of release etc.

In some embodiments according to above embodiments the sequencing is done at, greater than or equal to 60× or 40× sequence coverage. In some embodiments the sequencing of (ii) is low pass sequencing. In some embodiments, the low pass sequencing is less than 10×, less than 5×, less than 2.5×, less than 1×, or less than 0.5× coverage.

In some embodiments the subset of molecules is greater than or equal to 4 genome equivalents. In some embodiments the subset of molecules is <=1 genome equivalents.

In some embodiments a next generation sequencing (NGS) method is used in which individual molecules in the sample are tagged with unique identifier (UID) or barcode so that multiple samples can be processed simultaneously inside a sequencing or sequence detection device. This is useful when the fold coverage of sequencing is low and there is thus capacity in a flow cell to run multiple samples, hence saving on cost and time.

In some alternative embodiments, greater than 60× genome coverage us used which enables sampling of >90% of a human genome. However, this requires a larger amount of sample material and the cost of the test is greater because more molecules have to be analyzed.

In some embodiments of the above an in silico filter is applied before the molecular signature is determined. In some embodiments the filter comprises aggregating data only on loci that have previously been determined to have an association with cancer and removing data on loci that map to genomic loci where no association with cancer has previously been noted. In some embodiments other criteria for qualifying loci to be used for the molecular signature is applied.

In some embodiments loci with unexpected/abnormal change in methylation with respect to a background model of “normal” DNA comprising methylation data taken from many healthy samples, is aggregated.

In some embodiments the data on the methylation status that is aggregated is of loci in the genome where changes in methylation have previously been detected. In some embodiments these changes that have been previously detected are changes associated with cancer.

In some embodiments as well as determining the methylation status of each of the molecules mapped using a method for detecting absence or presence of methylation, the extent of methylation is also recorded. In some embodiments the extent of methylation of individual molecules is used to determine the molecular signature for cancer.

In some embodiments a clinical recommendation or decision regarding the management of the cancer is made based on the aggregated data and/or molecular signature.

In some embodiments based on the aggregated data and/or molecular signature, a clinical recommendation or decision regarding the presence, stage, tissue of origin, tissue of release of the cancer is made.

In some embodiments machine learning is used to determining the extent of methylation or the methylation of an individual molecule. In some embodiments machine learning, Bayesian or inference based algorithms are used to determining the extent of methylation or the methylation patterns of a sample. In some embodiments machine learning or Bayesian methods are used compose the molecular signature for cancer. In some embodiments machine learning or Bayesian methods are used to assist clinical decision making.

In some embodiments the sequence detection method is sequencing. In some embodiments the sequence detection method is oligonucleotide probing.

In some embodiments the method for detecting presence or absence of methylation comprises, enzyme digestion, antibody binding, protein binding, oligonucleotide binding, sequencing etc.

In some above embodiments, instead of isolating cfDNA from plasma, it is isolated directly from blood. In some embodiments, contamination of cellular fraction is tolerated.

In some embodiments the present disclosure comprises a method of detecting a signal for cancer from a drop of blood. In some embodiments, the non-nucleic components and blood cells within the blood drop are sequestered before performing the sequencing/sequence detection and methylation detection.

Some embodiments sample the genome randomly but then mine and filter the acquired data to look at the fraction (e.g. 10%) of all CpG sites within the genome that are identified as belonging to the set of sites universally hypomethylated among several cancer types. In an alternative embodiment of this the subset of molecules are not random, a subset of CpG (e.g. 10% of all CpG sites) within the genome that are identified as belonging to the set of sites universally hypomethylated among several cancer types, are pre-selected via enrichment (e.g. hybrid capture, CRISPR-based capture) in order to look at the methylation status at these sites. In some embodiments the set of sites universally hypomethylated among several cancer types, are pre-selected via enrichment.

In some embodiments, the present disclosure provides a composition comprising the set of CpG sites constituted from a method comprising: (i) taking a substantially complete set of CpG sites across the genome, (ii) testing each site to see if it fulfills a predefined criteria, and (iii) removing all sites that do not fulfill the predefined criteria. In some such embodiments the predefined criteria is that the site is hypomethylated in 70% of cancer cases, for which pertinent data is available and is hypomethylated in less than 30% of cases from healthy people for which pertinent data is available. In some embodiments the pertinent data is derived from the ENCODE database (see Table 1). In some embodiments the pertinent data is derived from data made available by Chan et al. (2013).

In some embodiments the composition comprises sequences used for enriching the CpG sites constituted in the above paragraph. In some embodiments any such sequence used for enrichment is designed to be >100 nt in length and cover at least one CpG site from the constituted set.

In some embodiments multiple modification types are detected. In some embodiments multiple modification types are not differentiated (e.g. hydroxymethylation is not differentiated from methylation). In some embodiments multiple modifications are differentiated. For example, hydroxymethyl cytosine, 5-methyl cytosine and non-modified cytosines are differentiated. In some embodiments, the extent of different modifications is determined. In some embodiments, the signal for cancer also takes into account sequence variants that are detected in the subset of molecules, as well as the modification status. For example, if the extent of sampling is not sufficient to cover every methylation site in the genome, it will concomitantly not be sufficient to cover every mutation in the genome of the sample. Nevertheless, a signal for cancer can be obtained by detecting a subset of possible mutations as well as a subset of methylation sites; in some embodiments the subsets may arbitrarily overlap from one sample to the next, but are not exactly the same. In some embodiments, single nucleotide polymorphisms or other types of polymorphisms (e.g. triplet repeats) that are determined in the sequence detection/sequencing are taken into account in obtaining a molecular signature for cancer; particular polymorphisms may be associated with a pre-disposition for certain types of cancer. In some embodiments the length of the molecules as well as the sequence or modification status is also determined, and this is also taken into account in determining the presence or absence of a signal for cancer.

In some embodiments the molecular signature for cancer is a signal for a type of cancer, a stage of cancer, or contains other information pertinent to cancer.

In some embodiments even though a random subset of modifiable sites are surveyed by the systems and method of the present disclosure. In some embodiments sufficient sites are surveyed to identify changes in molecular pathways, enabling insights into molecular mechanisms and targets for drug intervention to be identified.

In some embodiments the extent of the ˜28 million CpG sites in the genome that are surveyed is <50%, <10%, or <1%.

In some embodiments a molecular signature for a phenotype or disease other than cancer is obtained, by following the embodiments described above, but where the set of nucleic acids is obtained from individuals who have or are being checked for a particular disease and the predetermined criteria is derived from the methylation status along the molecules of reference or healthy individuals and, individuals who have the phenotype or disease.

In some embodiments where the molecules are attached to a surface or interface, each molecule within the subset is attached or fixed at a particular distinct location to which it remains fixed throughout the process of molecule identification and epigenetic modification detection.

In some embodiments the multiple signatures are obtained longitudinally (over 2 or more time-points) as the status or emergence of disease is tracked. The longitudinal information is used to make a clinical decision.

In some embodiments the data is compared to a database of methylation patterns obtained for different tissues. In some embodiments the data in the database is segregated into methylation patterns that are obtained for different cancer types. Thus, Tissue-specific or cancer-specific methylation information is used to determine if cell-free DNA from that tissue or cancer type is being shed into blood.

In some embodiments the molecular signature based on random sampling, is used to rule-out cancer. In some embodiments the molecular signature based on random sampling, is used to rule-in cancer or is a part of a triage approach in which further tests rule-in or rule-out cancer. The other approaches in the triage may include whole body imaging or targeted sequencing. In some embodiments the signature based on random sampling may be the first step in the triage. In some embodiments, a second round of sequencing or sequence detection may be used to confirm a positive signal for cancer from the first round. In some embodiments the second round of sequencing may start with targeted enrichment (where the first round has been random). In some embodiments the enrichment may be of a panel of cancer related genes or a whole exome.

In some embodiments the molecular signature provides a prediction, a predisposition or a diagnosis of cancer. In some embodiments the molecular signature may be of a phenotype or disease state or trait other than cancer.

Some embodiments of the present disclosure provide a method for determining the presence or absence of a phenotype in a subject comprising determining the state of modification of a subset of modifiable sites across the genome to yield a matrix of state likelihoods per corresponding site in the genome; comparing the matrix of state likelihoods per corresponding site in the genome determined for the current subject against a computer model of states per corresponding site in the genome that correspond to a specific disease state; determining the disease state (absence of, presence of, degree of) of the subject based on a threshold applied by the computer model. In some embodiments the modifiable sites are single or multiple-linked modifiable nucleotides. In some embodiments the multiple-linked nucleotides are those that form a haplotype along a contiguous stretch of the genome and may be represented in one or more cfDNA molecules. In some embodiments the multiple-linked nucleotides are those that form a functional association (e.g. as is the case of a suppressor with its target loci) and are from non-contiguous stretch of the genome and may be represented in one or more cfDNA molecules.

Some embodiments of the present disclosure provide a method for determining the presence or absence of a phenotype in a single cell comprising determining the state of modification of a subset of modifiable sites across the genome to yield a matrix of state likelihoods per corresponding site in the genome; comparing the matrix of state likelihoods per corresponding site in the genome determined for the current cell against a computer model of states per corresponding site in the genome that correspond to a specific cell phenotype; determining the phenotype state of the cell based on a threshold applied by the computer model.

Other embodiments are directed to systems, portable consumer devices, and computer readable media associated with methods described herein.

As disclosed herein, any embodiment disclosed herein when applicable can be applied to any aspect.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 illustrates a Venn diagram depicting relative size and overlap between sets of hypomethylated CpG sites among four unrelated samples in the ENCODE dataset. For each sample, the total number of hypomethylated sites and percentage of the total of CpGs that number represents is indicated. Percentages are among the 30% of CpG sites that satisfied a minimum read depth cut-off of 10 reads per site.

FIG. 2 illustrates the proportion of mapped bisulfite sequencing reads (WGBS) that were found to be methylated at the corresponding CpG sites along a region of Chromosome 2. The “Normal” track represents the average proportion of methylated reads across six healthy tissue samples. Each of the cancer tracks represent exact proportions for an individual sample. The red dotted lines mark “hypomethylated” sites: CpG sites that are hypomethylated with respect to the healthy cell population.

FIG. 3 illustrates hypothetical reads aligned to a reference sequence. Only CpG sites are depicted in the reference track A. Three read stacks spanning 3, 2 and 1 CpG sites respectively, taken from a cfDNA sample containing ctDNA at some small fraction (e.g. 0.01%) are aligned with reference track A. Reference track B is an exact copy of reference track A. There are three read stacks aligned to reference Track B, spanning the same CpG sites as in reference track A, but for a healthy cfDNA sample with no ctDNA

FIG. 4A illustrates a distribution of the hypomethylated reads measured for 100,000 tumor samples and 100,000 normal samples where the distributions of “hypomethylated” read counts are for reads that contain three contiguous biased CpG sites.

FIG. 4B illustrates a distribution of the hypomethylated reads measured for 100,000 tumor samples and 100,000 normal samples where the distributions of “hypomethylated” read counts are for reads that contain four contiguous biased CpG sites.

FIG. 4C illustrates a distribution of the hypomethylated reads measured for 100,000 tumor samples and 100,000 normal samples where the distributions of “hypomethylated” read counts are for reads that contain five contiguous biased CpG sites.

FIG. 5A illustrates 20,000 samples-worth of “hypomethylated” read counts (10,000 with 0.01% TF, vs 10,000 normals) plotted in three dimensions with one genome equivalent in accordance with an embodiment of the present disclosure. Each dimension is the total number of biased CpG sites spanned by the underlying reads.

FIG. 5B illustrates 20,000 samples-worth of “hypomethylated” read counts (10,000 with 0.01% TF, vs 10,000 normals) plotted in three dimensions with four genomes equivalent in accordance with an embodiment of the present disclosure. Each dimension is the total number of biased CpG sites spanned by the underlying reads.

FIG. 5C illustrates 20,000 samples-worth of “hypomethylated” read counts (10,000 with 0.01% TF, vs 10,000 normals) plotted in three dimensions with ten genomes equivalent in accordance with an embodiment of the present disclosure. Each dimension is the total number of biased CpG sites spanned by the underlying reads.

FIG. 5D illustrates 20,000 samples-worth of “hypomethylated” read counts (10,000 with 0.01% TF, vs 10,000 normals) plotted in three dimensions with forty genomes equivalent in accordance with an embodiment of the present disclosure. Each dimension is the total number of biased CpG sites spanned by the underlying reads. Numbers of biased CpG sites along the three axes can change as the number of genome equivalents increases. For example, at 40 genome-equivalents there is sufficiently large Poisson mean counts of reads spanning six sites that that set can be leveraged to widen the gap between the sample populations.

FIG. 6 is a flow diagram of example 1 in which the simulation is depicted as taking three phases. In phase 1 the background model of normal levels of methylation at each CpG site in the genome is built. In phase 2 each of the cancer sample methylation calls are compared against the background model to determine hypomethylated sites for each cancer. In phase 3 the process of discriminating between cfDNA samples containing no tumor DNA (ctDNA) versus samples that contain 0.01% ctDNA (0.01% tumor fraction) is simulated.

FIG. 7 illustrates a system architecture in accordance with an embodiment of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure is practiced without these specific details. In other instances, well-known methods, procedures, components, circuits, and networks have not been described in detail so as not to unnecessarily obscure aspects of the embodiments.

### Definitions

The terminology used in the present disclosure is for the purpose of describing particular embodiments only and is not intended to be limiting of the invention. As used in the description and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is construed to mean “when” or “upon” or “in response to determining” or “in response to detecting,” depending on the context. Similarly, the phrase “if it is determined” or “if [a stated condition or event] is detected” is construed to mean “upon determining” or “in response to determining” or “upon detecting [the stated condition or event]” or “in response to detecting [the stated condition or event],” depending on the context.

The term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Any aspect of the invention described for methylation detection can be applied to any type of epigenomic or epigenetic modification.

It will also be understood that, although the terms first, second, etc. is used herein to describe various elements, these elements should not be limited by these terms. These terms are only used to distinguish one element from another. For example, a first filter could be termed a second filter, and, similarly, a second filter could be termed a first filter, without departing from the scope of the present disclosure. The first filter and the second filter are both filters, but they are not the same filter.

As used herein, the terms “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The terms “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the terms “nucleic acid,” “nucleic acid molecule,” and “polynucleotide” are used interchangeably. The terms may refer to nucleic acids of any compositional form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing synthetic base analogs and or naturally occurring (epigenetically modified) base analogs, sugar analogs and/or a non-native backbone and the like), RNA/DNA hybrids and peptide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes as described herein (e.g., linear, circular, supercoiled, single-stranded, double-stranded and the like). In some instances, a nucleic acid is, or is from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A nucleic acid In some embodiments, can be from a single chromosome or fragment thereof (e.g., a nucleic acid sample from one chromosome of a sample obtained from a diploid organism). A nucleic acid molecule can comprise a complete length of a natural polynucleotide (e.g., a long non-coding (lnc) RNA, mRNA, chromosome, mitochondrial DNA or a polynucleotide fragment). A polynucleotide fragment can be at least 200 bases in length or can be at least several thousands of nucleotides in length, or in the case of genomic DNA, polynucleotide fragments can be hundreds of kilobases to multiple megabases in length.

In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of RNA or DNA synthesized, replicated or amplified from single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. In some embodiments, a nucleic acid is prepared using a nucleic acid obtained from a subject as a template.

As used herein, the terms “oligonucleotide” and “oligo” mean short nucleic acid sequences. In some embodiments, oligos are of defined sizes, for example, each oligo is k nucleotide bases (also referred to herein as “k-mers”) in length. Typical oligo sizes are 3-mers, 4-mers, 5-mers, 6-mers, and so forth. Oligos may also be referred to herein as N-mers.

As used herein, the term “label” encompasses a single detectable entity (e.g., wavelength emitting entity) or multiple detectable entities. In some embodiments, a label transiently binds to nucleic acids or is bound, either covalently or non-covalently to a probe. Different types of labels may blink during fluorescence emission, fluctuate in photon emission, or photo-switch off and on. Different labels is used for different imaging methods. In particular, some labels is uniquely suited to different types of fluorescence microscopy. In some embodiments, fluorescent labels fluoresce at different wavelengths and also have different lifetimes. In some embodiments, background fluorescence is present in an imaging field. In some such embodiments, such background is removed from analysis by rejecting a time window of fluorescence due to scattering or background fluorescence. If a label is on one end of a probe (e.g., a 3′ end of an oligo probe), accuracy in localization corresponds to that end of a probe (e.g., a 3′ end of a probe sequence and 5′ of a target sequence). Apparent transient, fluctuating, or blinking, or dimming behavior of a label can differentiate whether an attached probe is binding on and off from its binding site.

The term “imaging,” as used herein, includes both two-dimensional array and two-dimensional scanning detectors. In most cases, imaging techniques used herein will necessarily include a fluorescence excitation source (e.g., a laser of appropriate wavelength) and a fluorescence detector.

As used herein, the term “haplotype” refers to a set of variations that are typically inherited in concert. This occurs because a set of variations is present in close proximity on a polynucleotide or chromosome. In some cases, a haplotype comprises one or more single nucleotide polymorphisms (SNPs). In some cases, a haplotype comprises one or more alleles.

Several aspects are described below with reference to example applications for illustration. It should be understood that numerous specific details, relationships, and methods are set forth to provide a full understanding of the features described herein. One having ordinary skill in the relevant art, however, will appreciate that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in accordance with the present disclosure. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models in the present disclosure are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. Nearest neighbor models can be memory-based and include no model to be fit. For nearest neighbors, given a query point x₀ (a test subject), the k training points x₍ᵣ₎, r, . . . , k (here the training subjects) closest in distance to x₀ are identified and then the point x₀ is model using the k nearest neighbors. In some embodiments, Euclidean distance in feature space is used to determine distance as d₍ᵢ₎=∥x₍ᵢ₎−x_{(O)}∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Randomforest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (linear classifier) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter, “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of models and boosting. In some embodiments, an ensemble (two or more) of model is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of model is weighted or unweighted.

As used herein, the terms “model”, “regressor”, and “classifier” are used interchangeably.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n 500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×106, n≥5×106, or n≥1×107. As such, the algorithms, models, regressors, and/or classifiers of the present disclosure cannot be mentally performed. In some embodiments n is between 10,000 and 1×107, between 100,000 and 5×106, or between 500,000 and 1×106. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

The present disclosure exploits several key characteristics of methylation in cancer that are pertinent to monitoring and early screening efforts alike including: (1) Prevalence of hypomethylation in cancers at the single-nucleotide scale; (2) The relative diminutive hypomethylation in normal tissue of any type; (3) High level of conservation of site-specific hypomethylation across cancer types; (4) non-uniform distribution of hypomethylated sites across the cancer genome.

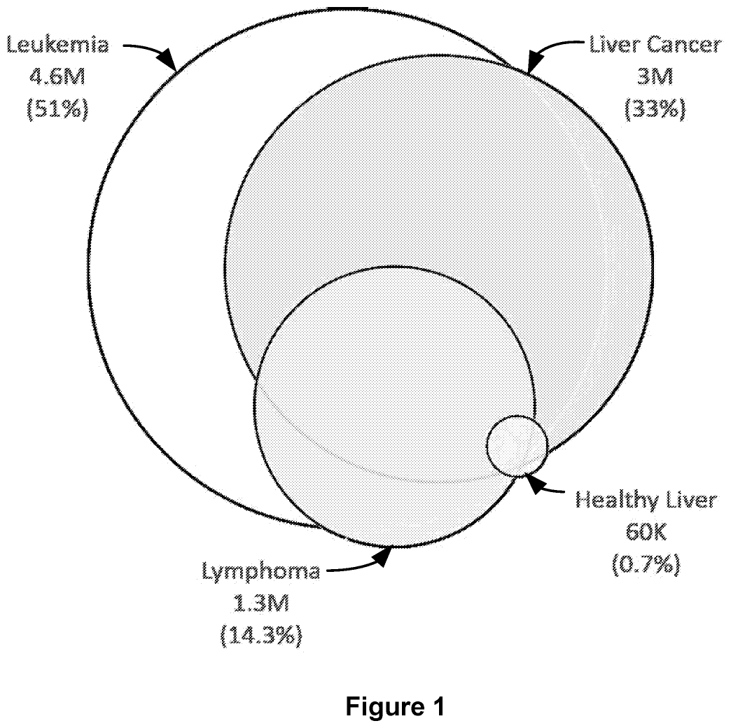

FIG. 1 shows a Venn diagram depicting relative size and overlap between sets of hypomethylated CpG sites among four unrelated samples in the ENCODE dataset. This figure illustrates the first three of four properties of methylation in cancer listed above. Table 1 lists the accession numbers for the underlying samples. This is a clear illustration of the similarity across cancer types. For each sample, the total number of hypomethylated sites and percentage of the total of CpGs that number represents is indicated. Percentages are among the 30% of CpG sites that satisfied a minimum read depth cut-off of 10 reads per site.

The non-uniform distribution of CpG's across the genome results in many regions of localized higher density. For example, there are many more regions of length <=200 bp in the genome that contain 3 or more CpG's than expected by random chance. Furthermore, aberrant methylation patterns also appear to possess local biases in the genome resulting in local concentrations of either hypomethylated or hypermethylated CpG sites. These localized aberrations have been shown to be cancer and tissue specific.

These priors allow for filtering out molecule sequences that do not span any sites previously observed to be hypomethylated in a cancer type of interest

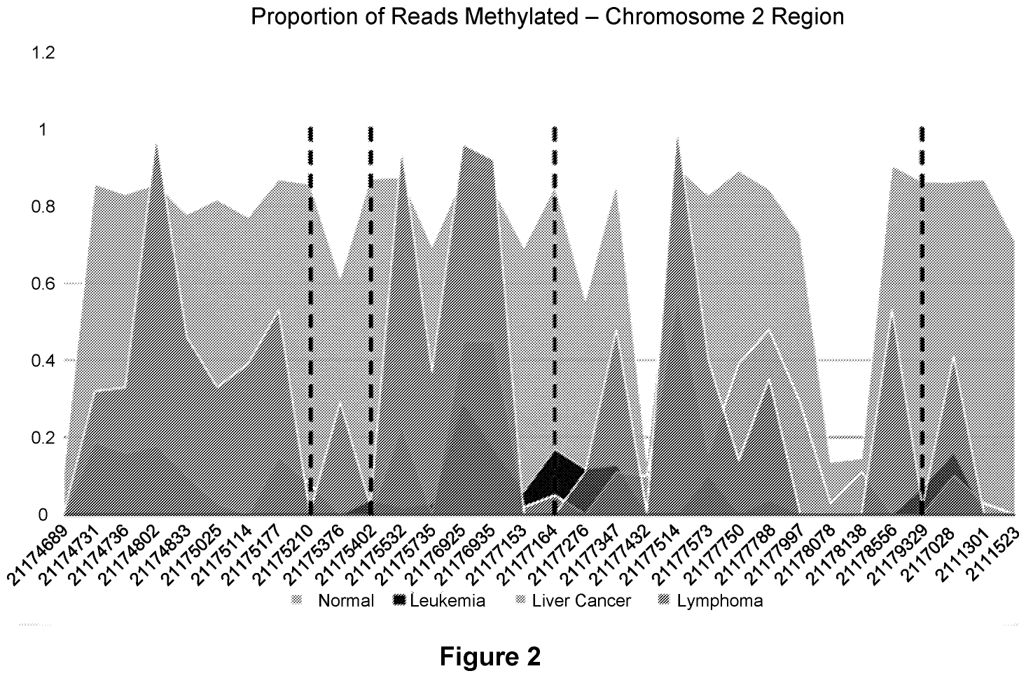

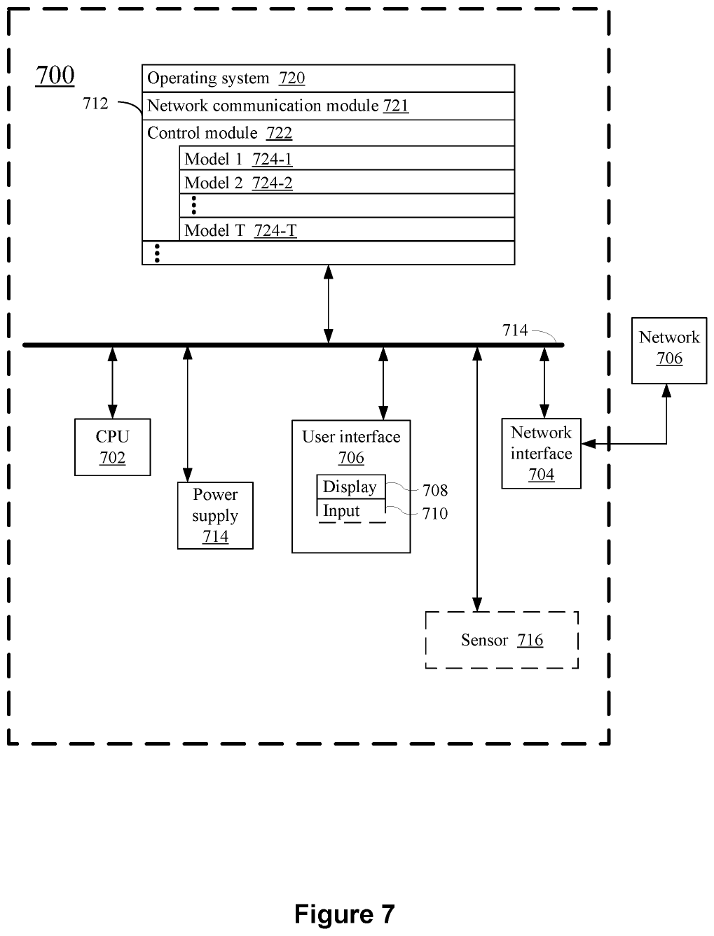

FIG. 2 illustrates the proportions of reads that showed methylation at each of roughly 100 CpG sites found within a 7 kb region of Chromosome 2 for the samples listed in Table 1. It is clear from the figure that the degree of methylation is starkly contrasted between healthy and cancerous cells. The four dotted lines in FIG. 7 mark examples of CpG's that were found to be hypomethylated sites across all four cancer samples analyzed from Table 1. Roughly 10% of all CpG sites within the genome belong to this set of sites universally hypomethylated among these cancer samples which is a 30-fold larger proportion than expected by random chance. All four samples were derived from unrelated individuals and unrelated cancer types.

Some embodiments of the present disclosure provide models of expected methylation patterns across both healthy and cancerous cells. These models can be derived from any combination of whole genome bisulfite sequencing data, bead array data, targeted sequencing data or direct single molecule data (ONT, PacBio, XGenomes). These models are used to assign a likelihood that any given CpG site will be methylated or not, given the state of the sample (healthy or cancerous) as well as the tissue of origin for any individual molecule.

In some embodiments, molecules are identified by mapping them to a reference genome. After the molecules have been mapped to a reference genome, each mapped genomic locus comprises the number of molecules sampled from the Poisson mean coverage depth. For example, if 72 million cfDNA molecules of 165 bp average length are sequenced, then that approximates to four genome-equivalents being measured. FIG. 8 depicts this post-mapping strategy. There are six different mapped read stacks in the figure (numbered 1-6). Three of the six (set A) represent molecules sequenced from a cfDNA sample containing 0.01% tumor fraction. The remainder (set B) represent molecules that span the same loci as in set A but for a healthy cfDNA sample without any circulating tumor DNA.

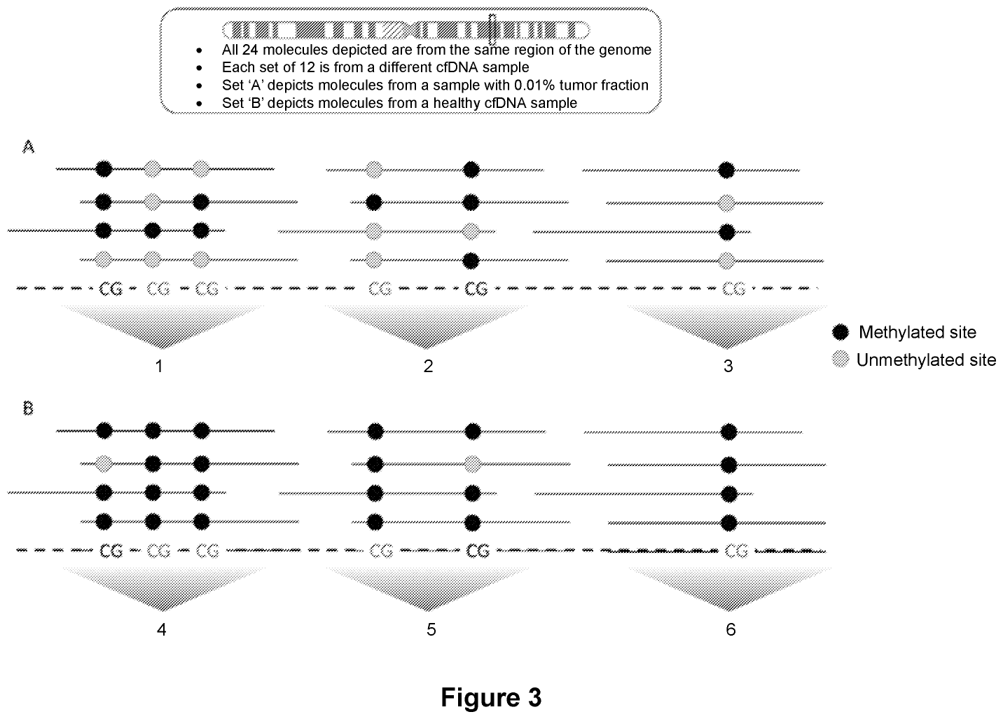

Models that capture site-specific methylation likelihoods are used to generate a list of CpG sites that are expected display some type of aberrant methylation in the genome given some other property of the sample such as disease state and tissue of origin. These priors allow for filtering out molecule sequences that do not span any sites previously observed to be hypomethylated in a cancer type of interest, for example. In FIG. 3, all reads have passed the hypomethylation filter, meaning that each read stack spans at least one site known to be biased towards hypomethylation in the cancer type in question.

In several embodiments of the present disclosure, one metric of interest is the number of molecules that span at least one known biased site which are also hypomethylated across all biased sites spanned by that same molecule. For example, in read stack A-3 two of the four reads are entirely unmethylated and in stacks A-1 and A-2, one of the reads is entirely unmethylated. Therefore, four reads depicted in (A) satisfy this criterion. In contrast, none of the reads in (B) pass this test. Note that all read stacks illustrated in FIG. 3 contain at least one biased site, but some contain additional, unbiased CpG sites.

Some embodiments of the present disclosure break out sequence reads based on the total number of biased CpG sites contained therein. The presence or absence of bias is determined by a model of expected aberrations derived from comparison of modification status between healthy and affected populations. For example, some CpG sites may be methylated in less than 30% of all molecules derived from all cancerous cells while those same sites are methylated in greater than 70% of all molecules derived from normal cells. In some embodiments, this type of cohort bias forms the basis of an expectation for the general population that has yet to be observed.

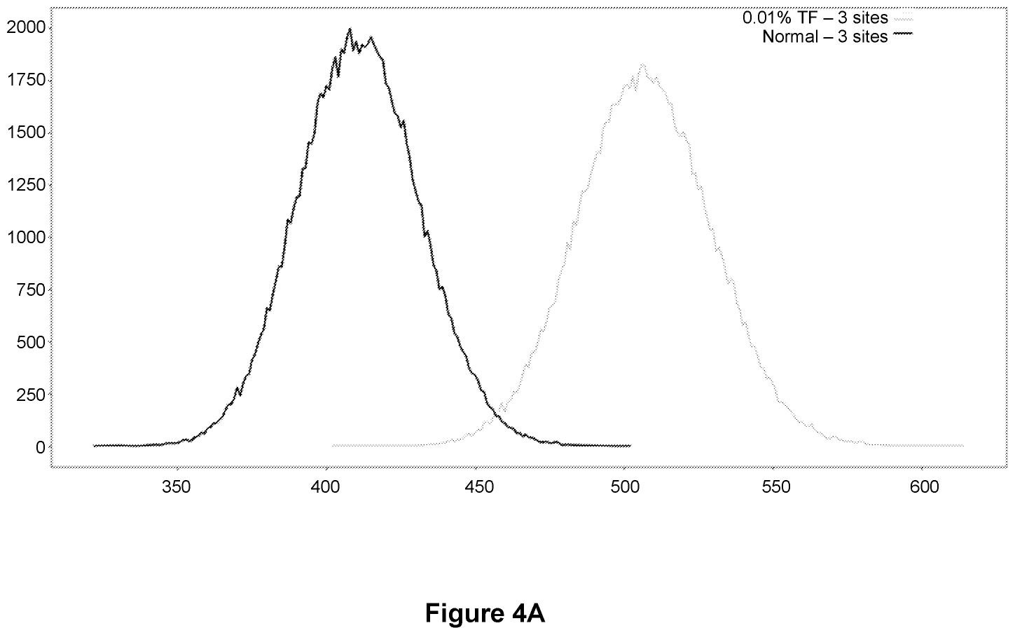

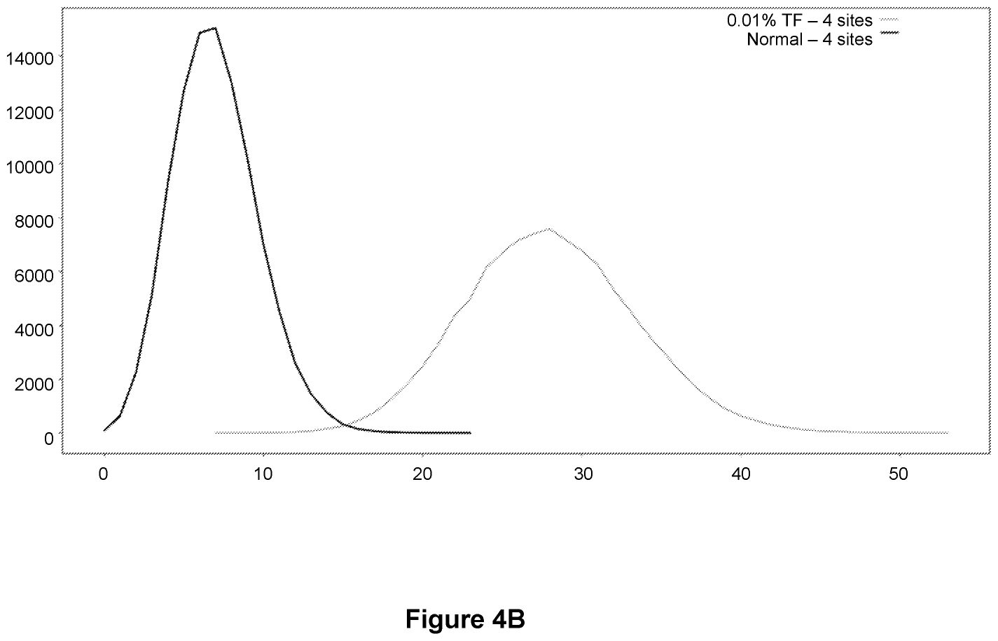

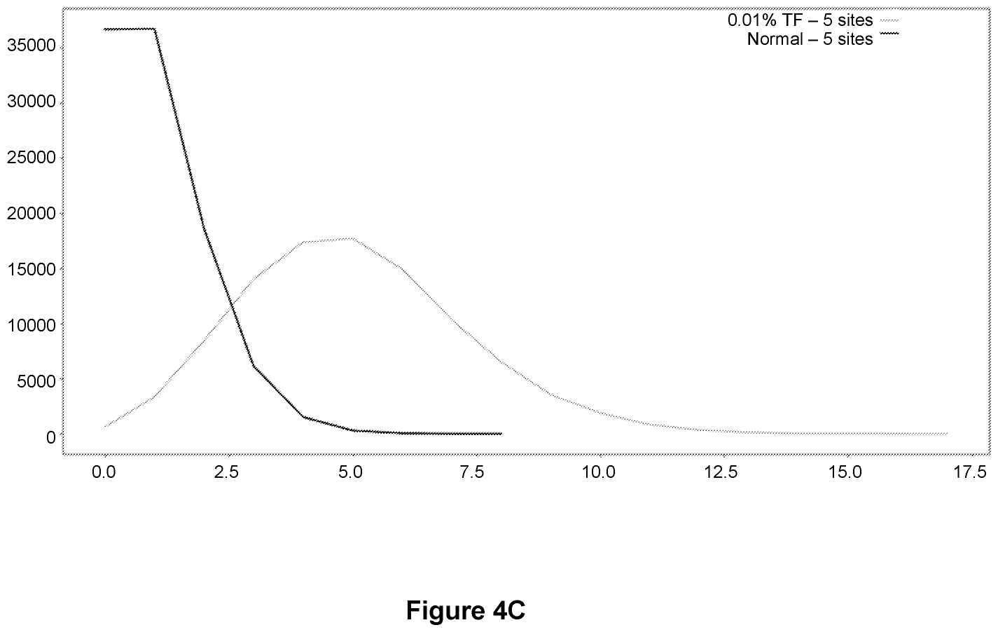

Some embodiments of the present disclosure segregate molecules sequenced from a sample that are predicted, by mapping to the genome, to contain one, two, three or more such cohort biased CpG sites. Such embodiments further count the number molecules observed to be nonmethylated at all the cohort biased sites contained in that molecule, again segregated by total number of expected biased sites. FIG. 4 illustrates how these counts would differ between molecules taken from a healthy plasma sample and those taken from a plasma sample containing 0.01% tumor fraction (e.g., 0.01% of cfDNA molecules in the plasma originated in cancerous cells). In the figure, a histogram appears for each of three different categories of molecules each category represented in both ctDNA-free (e.g., healthy) and ctDNA-containing cfDNA. Each category is defined by the number of cohort biased sites contained (three, four, or five) in those molecules, as predicted by mapping the molecules to a reference genome and looking for CpG sites in that genomic region found to be biased in the models described above. Additional embodiments comprise a larger number of categories to include molecules that contain one, two, three or more such cohort biased sites up to the limit of what was observed in the sample. Note that in every hypothetical sample, four genome equivalents worth of cfDNA is assumed to be measured thus allowing for direct comparison of absolute counts for illustration purposes.

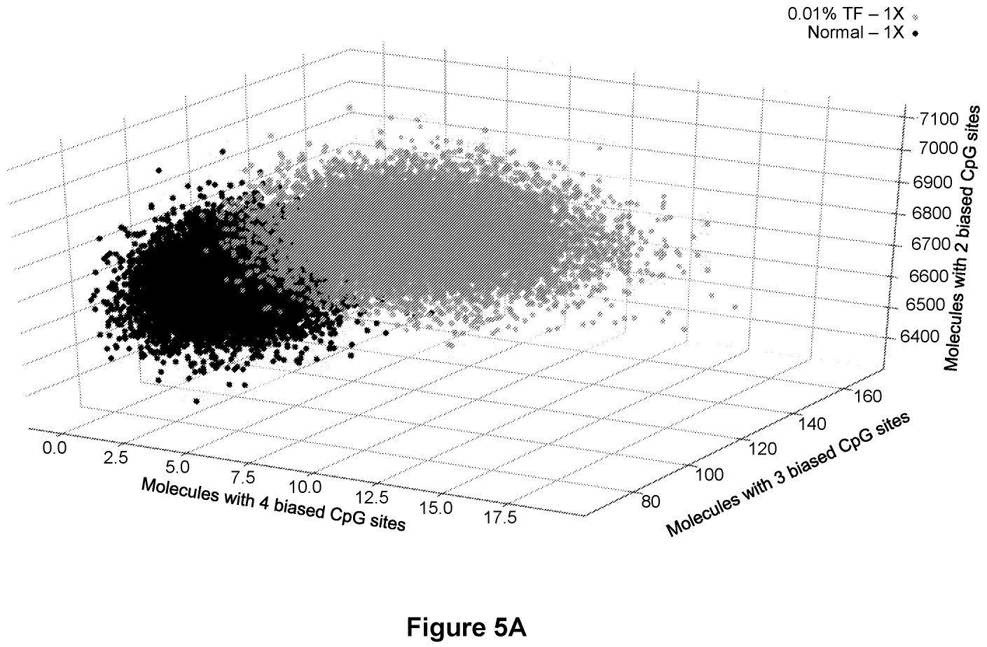

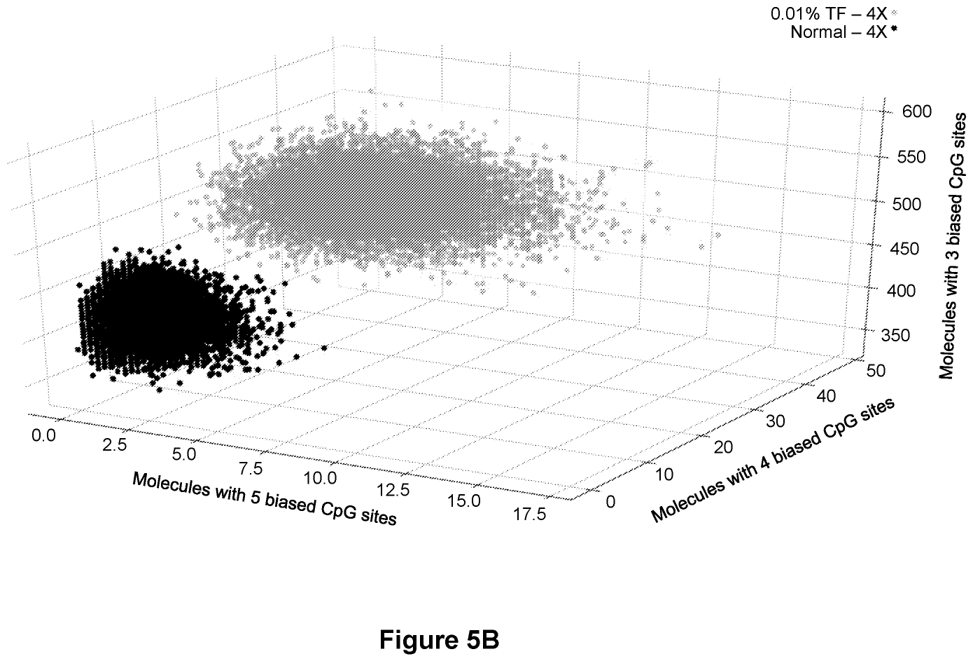

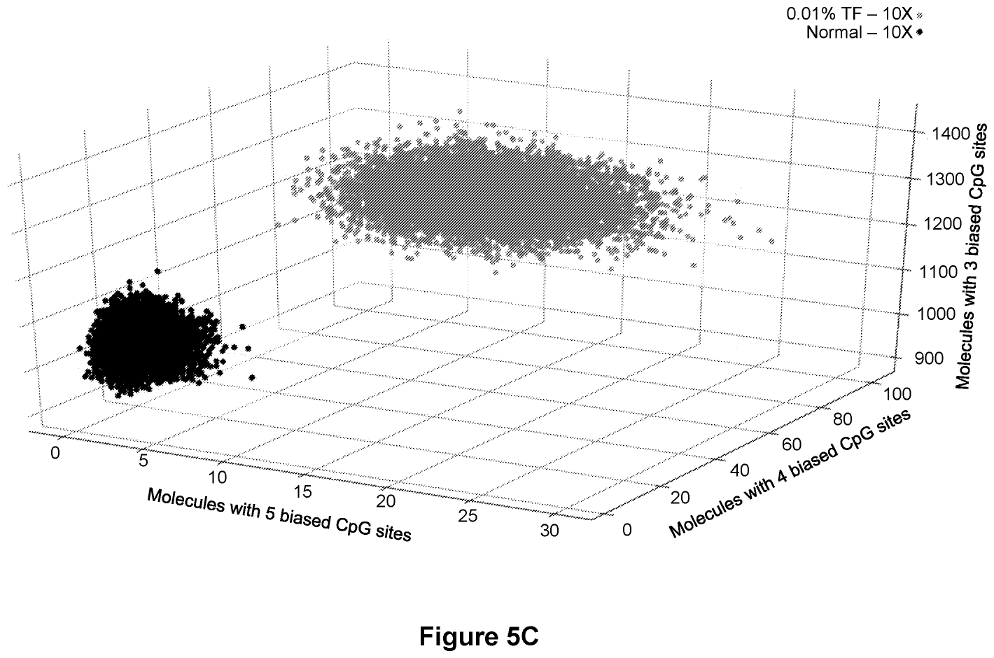

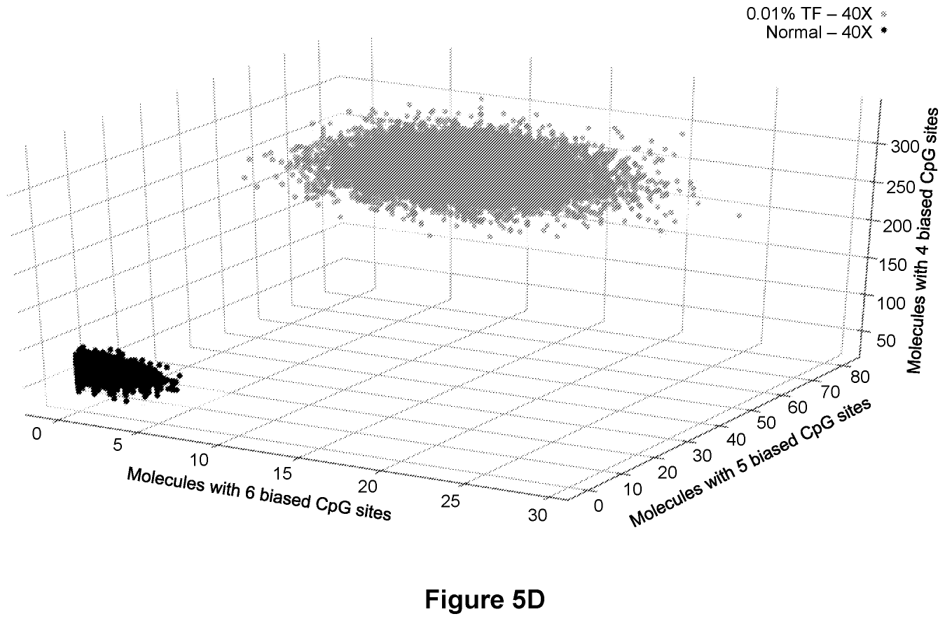

In FIG. 4 each category of molecule is shown to clearly segregate as a function of sample-type (healthy vs cancerous) between the distributions of molecule counts and could be used as the basis of a one-dimensional discriminator between the two sample populations. However, each subset of molecules (e.g. those containing three, four or five biased sites) is independent of the others. In some embodiments of the present disclosure, a plurality of subsets of molecules are used to generate a high-dimensional discriminator between the two sample populations. The effects of taking this step are illustrated in FIG. 5. In the figure, the two sample populations are depicted in three dimensions, specifically the molecule counts for the 3-biased-site, 4-biased-site and 5-biased-site molecules. Note that with four or more genome equivalents, cancer and normal populations are extremely well separated (zero overlap among 20,000 samples total). Classification in this three-dimensional space can be accomplished using any of several linear methods (LDA, regression, support vectors) are 99.99% accurate. Only three dimensions are portrayed herein for purposes of clarity. Several embodiments classify samples using one, two, three, or more such dimensions.

### Exemplary System Embodiments

Details of an exemplary system are now described in conjunction with FIG. 7. FIG. 7 is a block diagram illustrating a system 100 in accordance with some implementations. Device 700 in some implementations may include one or more processing units (CPU(s)) 702 (also referred to as processors or processing core), one or more network interfaces 706, a user interface 706, a memory 712, and one or more communication buses 714 for interconnecting these components. The one or more communication buses 714 optionally include circuitry (sometimes called a chipset) that interconnects and controls communications between system components. Memory 712 typically includes high-speed random access memory, such as DRAM, SRAM, DDR RAM, or lower speed memory such CD-ROM, digital versatile disks (DVD) or other optical storage, magnetic cassettes, magnetic tape, magnetic disk storage or other magnetic storage devices, magnetic disk storage devices, optical disk storage devices, ROM, EEPROM, flash memory devices, or other non-volatile solid state storage devices. In some embodiments, memory 712 optionally includes one or more storage devices remotely located from CPU(s) 102. In some embodiments, memory 712 comprises non-transitory computer readable storage medium. In some implementations, memory 71 stores the following programs, modules and data structures, or a subset thereof, sometimes in conjunction with persistent memory 112:
an optional operating system 720, that includes procedures for handling various basic system services and for performing hardware dependent tasks;
a network communication module 721 for communication across network 706; and
a control module 722 for determining whether a test subject has a phenotype, where the control module makes use of one or more model 724.

In various implementations, one or more of the above identified elements are stored in one or more of previously mentioned memory devices, and correspond to a set of instructions for performing a function as described hereinabove. Herein, above identified modules, data, or programs (e.g., sets of instructions) need not be implemented as separate software programs, procedures, datasets, or modules, and thus various subsets of these modules and data is combined or otherwise re-arranged in various implementations. In some embodiments, one or more of the above identified elements is stored in a computer system, other than that of system 100, that is addressable by system 100 so that system 100 may retrieve all or a portion of such data when needed.

Examples of network communication modules 721 include, but are not limited to, the World Wide Web (WWW), an intranet, a local area network (LAN), controller area network (CAN), Cameralink and/or a wireless network, such as a cellular telephone network, a wireless local area network (WLAN) and/or a metropolitan area network (MAN), and other devices by wireless communication. Wired or wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.1 in), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Although FIG. 7 depicts a “system 700,” the figure is intended more as functional description of the various features that is present in computer systems than as a structural schematic of the implementations described herein. In practice, and as recognized by those of ordinary skill in the art, items shown separately could be combined and some items could be separated.

### EXAMPLES

### Example 1

Summary Disclosed are simulations based on ENCODE data that show obtaining a signal for cancer from a very small amount of sample material at very low tumor fraction by looking at methylation haplotypes on cell-free DNA comprising CpG sites that have previously been shown to become hypomethylated in cancer.

Abstract. This example demonstrate how the disclosed methods leverage the nature of cancer methylomes for liquid biopsy. Several characteristics of methylation in cancer that are pertinent to monitoring efforts are shown. These include (1) high prevalence of hypomethylation (loss of methylation) in cancers at the single-nucleotide scale; (2) low prevalence of hypomethylation in normal tissue of any type; (3) high level of conservation of site-specific hypomethylation across cancer types; and (4) non-uniform distribution of hypomethylated sites across the cancer genome. This example derives a model of these characteristics from whole genome bisulfite sequencing (WGBS) results published through the ENCODE consortium. This model is then used as a basis for simulating large populations of cfDNA samples representing normal plasma and those containing trace amounts (0.01%) of circulating tumor DNA. This example demonstrates the accurate discrimination of these two populations under assumptions (see below) that utilize the ability of the disclosed platform to sample the methylation status of CpG sites across the genome. The simulations suggest the disclosed approach can detect a signal for any cancer from a small amount of blood containing as few as four genome equivalents, even when the tumor fraction is <0.01%.

A superior approach for a proven metric in cancer monitoring. FIG. 1 illustrates that abnormal methylation is a hallmark of cancer and the patterns are conserved across cancer types. FIG. 1 is a Venn diagram depicting relative size and overlap between sets of hypomethylated CpG sites among four unrelated samples in the ENCODE dataset. For each sample, the total number of hypomethylated sites and percentage of the total of CpGs that number represents is indicated. Percentages are among the 30% of CpG sites that satisfied a minimum read depth cut-off of 10 reads per site. In some embodiments, methylated detection and sequencing makes use of techniques disclosed in U.S. Pat. Nos. 10,982,260; 11,061,013; and 11,066,701, as well as U.S. patent application Ser. No. 16/245,929, each of which is hereby incorporated by reference that, in contrast to competitors' methods, does not rely on Illumina sequencing nor bisulfite treatment. In some embodiments, the disclosed systems and methods, making use of the techniques disclosed in U.S. Pat. Nos. 10,982,260; 11,061,013; and 11,066,701, as well as U.S. patent application Ser. No. 16/245,929, directly detect the genomic identity of individual molecules of DNA and determine the methylation status of CpG sites thereon. The disclosed systems and methods collect data from a sufficient number of molecules (as discussed in this example) to detect a signal for cancer.

In some embodiments, the XGenomes optical super-resolution sequencing approach that utilizes single molecule localization algorithms is capable of detecting 10⁸-10⁹ molecules on a state-of-the-art 5-million-pixel CMOS sensor. See, U.S. Pat. Nos. 10,982,260; 11,061,013; and 11,066,701, each of which is hereby incorporated by reference. The amount of cfDNA contained in a 10 mL blood sample (of which 6 mL is plasma) is approximately 4,000 to 6,000 genome equivalents on average. This example shows how, using the disclosed systems and methods, only four genome equivalents—72 million molecules—are needed to obtain a signal for cancer. The sequencing methods disclosed in in U.S. Pat. Nos. 10,982,260; 11,061,013; and 11,066,701, as well as U.S. patent application Ser. No. 16/245,929, are well-capable of detecting this number in a single field of view. This is the amount of DNA in less than a single drop of blood (˜50 ul).

The disclosed systems and methods avoid common pitfalls and exceeds existing methods in a number of ways. The fact that disclosed systems and methods do not amplify (and hence bias or corrupt) the sample, using the techniques disclosed in U.S. Pat. Nos. 10,982,260; 11,061,013; and 11,066,701, and looks at single molecules directly makes it high resolution-capable of discriminating methylation haplotypes on individual cfDNA molecules- and exquisitely sensitive. The sensitivity is further enhanced because the test can utilize any combination of CpG methylation sites in the genome to detect a signal for cancer. Moreover, it requires no prior curation of mutations in an individual's tumor by sequencing, it makes no presumptions about which part of the genome to target (hence requires no enrichment) and is tumor-agnostic or pan-cancer, e.g., it is exactly the same test for all tumor types. The accuracy, ease, speed, and economics of the technology make it ideal for minimal residual disease (MRD) detection, frequent recurrence monitoring as well as for future cancer screening applications.

The Nature of the problem. Changes in methylation status of CpG sites are widespread among cancer genomes (See, Vidal et al., 2017, “A DNA methylation map of human cancer at single base-pair resolution,” Oncogene 36, 5648-5657). Methylation patterns are shown to be tissue and cancer specific (Vidal, Id.). At its core, the disclosed systems and methods is looking for loci within the genome where there is an unexpected change in methylation with respect to a background model of “normal” DNA comprising methylation data taken from many healthy samples. This example focuses on hypomethylation (loss of methylation) patterns as they are widespread among cancer genomes (Gama-Sosa et al., 1983, “The 5-methylcytosine content of DNA from human tumors,” Nucleic Acids Res. 11:6883-6894; and Feinberg and Vogelstein, 1983, “Hypomethylation distinguishes genes of some human cancers from their normal counterparts,” Nature 301, pp. 89-92 and are well represented in the ENCODE project, which we utilize here as a basis for our simulations. In this example a potential “methylation site” is defined as any genome location containing a CpG. Further, any site is considered “hypomethylated” in a sample if less than 30% of the sequencing reads show methylation where that same site showed greater than 70% methylation among the reads taken from the healthy tissue samples. We refer to these hypomethylated CpGs as “Hypomethylation-Biased”.

All the data underlying this example were obtained from whole genome bisulfite sequencing (WGBS) experiments deposited in the ENCODE project repository. See ENCODE integrative analysis (PMID: 22955616; PMCID: PMC3439153) and the ENCODE portal (PMID: 31713622; PMCID: PMC7061942).

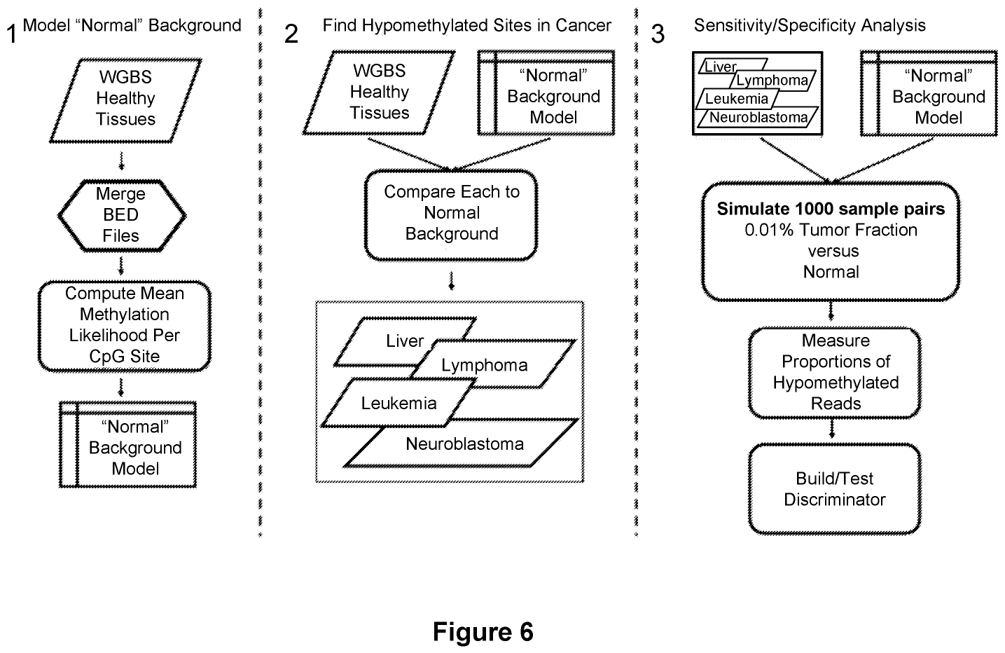

Table 1 lists the ENCODE accession numbers, tissue types and degree of hypomethylation observed for each of 10 samples. The six healthy samples (first six entries in Table 1) were used to build the background model of methylation across the genome (see FIG. 6, phase 1).

TABLE 1
A listing of the WGBS data taken from the ENCODE
repository to form the basis of this example.

| ENCODE Accession | Tissue Type | Percent Hypomethylated |
|---|---|---|
| ENCSR579AXB | Heart | 0.75 |
| ENCSR108ESU | Liver | 0.7 |
| ENCSR556KEJ | Lung | 0.8 |
| ENCSR781LIC | Pancreas | 0.65 |
| ENCSR601MHU | Thyroid | 0.9 |
| ENCSR267SNS | Stomach | 1 |
| ENCSR765JPC | Leukemia | 51 |
| ENCSR881XOU | Liver Cancer | 33 |
| ENCSR999CXD | Lymphoma | 14.3 |
| ENCSR145HNT | Neuroblastoma | 12.5 |

Prior to running the simulation, the proportion of CpG sites that were methylated among the samples taken from the ENCODE project were analyzed. Those proportions are summarized in FIG. 1 which shows a Venn diagram of hypomethylated sites found in each of 4 cancer samples table 1 and one healthy tissue (ENCSR108ESU) from table 1. Note that for all steps in this example, only CpG sites that contained sufficient read depth (>=10 reads) across all 10 samples were included. Roughly 30% of CpG sites, or 10 million sites in total, satisfied this entrance criterion.

FIG. 1 further illustrates the stark contrast between healthy and cancerous cells. Note further that there is roughly 9000 overlap (with respect to liver cancer) between the leukemia and liver cancer samples while there is less than 2% overlap between healthy liver and liver cancer. Both of those percentages are larger than expected by random chance. However, by this measure, tumor cells from any tissue type clearly have more in common with one another than with any healthy cells.

It is to be emphasized each of these samples were taken from unrelated individuals. The degree of overlap in the hypomethylated sites among the cancer samples is roughly six-fold larger than expected by random chance. This unexpectedly large effect suggests that the number of tumor-specific hypomethylated sites might not significantly increase as more methylation data for samples of the same cancer type are collected.

FIG. 2 shows the proportions of reads that showed methylation at each of roughly 100 CpG sites found within a 7 kb region of Chromosome 2. It is clear from the figure that the degree of methylation is starkly contrasted between healthy and cancerous cells. The four dotted red lines in FIG. 3 mark examples of CpG's that were found to be hypomethylated sites across all three cancer samples plotted here. Roughly 10% of all CpG sites within the genome belong to this set of sites universally hypomethylated among the cancer samples. In more detail, FIG. 2 show the proportion of mapped bisulfite sequencing reads (WGBS) that were found to be methylated at the corresponding CpG sites along a region of Chromosome 2. The “Normal” track represents the average proportion of methylated reads across 6 healthy tissue samples. Each of the cancer tracks represent exact proportions for an individual sample. The red dotted lines mark “hypomethylated” sites: CpG sites that are hypomethylated with respect to the healthy cell population in all three cancer genomes (each of different cancer types) plotted here.

Making optimal use of a scarce resource. Using sequencing techniques disclosed in U.S. Pat. Nos. 10,982,260; 11,061,013; and 11,066,701, millions of individual molecules taken from the sample are directly measured, without any enrichment, instead downstream selection of relevant mapped reads in silico is relied upon. After reads have been mapped to a reference genome (e.g., a human reference genome), each mapped locus comprises the number of molecules sampled from the Poisson mean coverage depth. For example, if 72 million cfDNA molecules of 165 bp average length are sequenced, then that approximates to 4 genome-equivalents being measured. FIG. 3 depicts this post-mapping strategy. There are 6 different mapped read stacks in the figure (numbered 1-6). Three of the 6 (set A) represent molecules sequenced from a cfDNA sample containing 0.01% tumor fraction. The remainder (set B) represent molecules that span the same loci as in set A but for a healthy cfDNA sample without any circulating tumor DNA. Recall from Phase 2 of the flow diagram in FIG. 6, there is a preprocessing step in which each cancer sample's WGBS data is compared to the normal background model of methylation distributions obtained in phase 1. This yields a list of hypomethylated CpG sites in the genome per cancer type/sample. This prior allows for the filtering out of reads that do not span any sites previously observed to be hypomethylated in that cancer type. In FIG. 3, all reads have passed the hypomethylation filter, meaning that each read stack spans at least one site known to be biased towards hypomethylation (a ‘biased’ site) in the cancer type in question.

One metric of interest is the number of reads that span at least one known biased site that are hypomethylated across all biased sites spanned. For example, referring again to FIG. 3, in read stack A-3 two of the four reads are entirely unmethylated and in stacks A-1 and A-2, one of the reads is entirely unmethylated. Therefore, 4 reads depicted in (A) satisfy this criterion. In contrast, none of the reads in (B) pass this test. Note that all read stacks illustrated in FIG. 3 contain at least one biased site, but some contain additional, unbiased CpG sites. In the final analysis, reads are segmented based on the total number of biased CpG sites spanned.

Cancer-specific discrimination. In phase 3 in accordance with FIG. 6, the potential of the disclosed systems and methods to discriminate between samples containing trace amounts of ctDNA and those without was analyzed. Thousands of genome-scale simulations were performed based on the normal background model and lists of hypomethylation-biased sites in each cancer type.

In each simulation, a random 4-fold coverage of each genome was generated in silico. The methylation status at each CpG site was randomly sampled from a normal distribution modeled on the real samples processed in phases 1 and 2. Only those reads that were known to span at least one hypomethylation biased site were analyzed. For example, when running a simulation where detection of trace amounts of ctDNA from liver cancer was being sought, reads that spanned any one of the 33% of hypomethylated sites observed in the ENCODE data (depicted in FIG. 1) were sought. For every read that spanned at least one biased site, a determination was made as to whether they were entirely hypomethylated among all of the biased sites spanned. Reads were further segmented based on the total number of biased sites they spanned with a minimum of 1 site and a maximum of 10 sites, expanding upon what is depicted in FIG. 3. For each population of reads, segmented by number of biased sites, a determination was made of the number of “hypomethylated reads”, i.e. those that are hypomethylated across all sites expected to be biased towards hypomethylation in the tumor.

These statistics were computed across 200,000 simulated samples. The resulting distributions of hypomethylated read counts were plotted in FIG. 4. For example, in FIG. 4A, the blue and yellow histograms show the hypomethylated counts from among the reads that contained exactly 3 biased CpG site for normal and ctDNA-containing samples, respectively. Similar histograms appear in FIGS. 4B and 4C for reads containing 4 and 5 biased sites, respectively.

Since each of the read populations plotted in FIG. 4 are independent, a determination can be made as to how well the sample populations segregate in higher dimensions. The results are plotted in FIG. 5. The various plots of FIG. 5 vary the number of genome equivalents (from 1 to 40) to show the impact on separation between the sample populations.

Four or more genome equivalents cancer and normal populations are extremely well separated (zero overlap among 20,000 samples total). Classification in this three-dimensional space is straightforward, where any of several linear methods (LDA, regression, support vectors) are 99.99% accurate.

This example makes use of the observation that a single hypomethylated CpG in non-tumor derived cell-free DNA is relatively rare, but hypomethylation at CpG sites in tumor derived DNA is relatively common. So even at very low tumor fraction, when one detects a molecule with a large number of hypomethylated CpG, the surprisal value is high suggesting that it is indeed a rare tumor-derived molecule. Current liquid biopsy strategies are inherently mismatched to the requirements of measuring exceedingly rare targets in an unbiased way without adding high cost or high complexity. For example, hybrid capture strategies attempt to reduce sample complexity up-front by selecting a narrow set of predetermined loci (Liu et al., 2020, “Sensitive and specific multi-cancer detection and localization using methylation signatures in cell-free DNA,” Annals of Oncology 31(6)). Unfortunately, in any early screening or MRD scenario, it is likely that the tumor fraction of the sample will be extremely low (<=0.01%). Each bait in the capture panel then has a 1 in 10,000 chance of finding its locus within the tumor fraction. This necessitates broad expansion in the number of baits in the panel to far larger than 10,000 just to have the chance of seeing at least a small number of ctDNA molecules. Fortunately, targeted enrichment can be avoided using the sequencing techniques disclosed in U.S. Pat. Nos. 10,982,260; 11,061,013; and 11,066,701, as well as U.S. patent application Ser. No. 16/245,929. Because the number of methylation sites to be detected is plentiful, and the disclosed systems and methods can detect any site in the genome, the disclosed systems and methods do not need to look at specific sites in the genome. It therefore does not require target enrichment. The disclose model utilizes information on whether a hypomethylated CpG site has been correlated with cancer before but this is done in silico after detection rather than through prior selection. What is particularly compelling is that the disclosed systems and methods can provide an answer irrespective of which sites are represented in any particular 4 genome-equivalents from a patient sample. Remarkably, 4 genome equivalents are represented within a single 50 uL blood droplet.

The simplicity, low cost and small form-factor of the disclosed systems and methods will allow for the scaling within centralized (CLIA) scenarios and ultimately in near-patient (IVD) settings. The disclosed systems and methods provides a feasible solution for frequent cancer monitoring following diagnosis or treatment because it does not require access to solid tumor to first develop a personalized assay and it can be conducted at low cost and a turnaround time that is easily within one day. The approach could also be applied to early cancer detection in asymptomatic individuals which opens up the prospect of large-scale cancer screening.

Assumptions. The calculations in this example assume i) perfect mapping of reads, ii) perfect calling of degree of hypomethylation, iii) minimum 4-fold coverage of the genome that is not biased away from sites of interest; and iv) that the model will not break down when many samples have been observed. Perfect mapping is realistic since only a small percentage of the genome is needed, and the sequencing library can be biased accordingly. Perfect calling of degree of hypomethylation is realistic by making use of oversampling for every molecule. Each of the methylation probes is >99% accurate on its own. Each site is interrogated by several such probes, and there are sets of measurements across several sites per molecule. The lack of bias away from the sites of interest is assumed to be superior to that observed in the WGBS data downloaded from ENCODE since the disclosed systems and methods do not require any amplification steps nor any bisulfite modifications. The robustness of the model to large numbers of samples is assumed due to the relative scarcity of hypomethylation among all of the normal samples observed as well as the enormous overlap in hypomethylated sites among such a disparate collection of unrelated cancer samples.

### Example 2

Disclosed is the process for conducting bisulfite sequencing of plasma derived cell-free DNA that is used as a basis for downstream selection of relevant mapped reads in silico to detect the methylation states that are a basis for detecting a signal for cancer using a method such as described in Example 1.

Isolation of a random set of cell free DNA molecules. 10 ml blood sample was collected from subject in a Streck-BCT tube (Streck, Omaha, NE). Plasma was isolated from sample by centrifuged at 3000×g for 15 min at 4° C.). cfDNA was extracted from 5 ml of plasma using the QIAamp circulating nucleic acid kit ((Qiagen, Valencia, CA). Total DNA was quantified using a Qubit dsDNAHS Assay kit (Life Technologies, Grand Island, NY, USA).

Bisulfite Library preparation of a random set of cell free DNA molecules. DNA was subjected to end repair, mono-adenylation, and ligation. Ligated products were treated with sodium bisulfite (EpiTect; Qiagen) using a cycling incubation of 95° C. for 5 min, 60° C. for 25 min, 95° C. for 5 min, 60° C. for 85 min, 95° C. for 5 min, and 60° C. for 175 min followed by three cycles of 95° C. for 5 min, 60° C. for 180 min. Each reaction was purified according to the manufacturer's instructions (Qiagen). Converted product was amplified using Pfu Turbo Cx Hotstart DNA polymerase (Agilent) and the TruSeq primer cocktail (Illumina) using the following cycling parameters: 95° C. for 5 min; 98° C. for 30 s; 14 cycles of 98° C. for 10 s, 65° C. for 30 s, 72° C. for 30 seconds; and 95° C. for 5 minutes.

Indexing each random set of cellfree DNA molecules samples. WGBS sequencing libraries prepared from 10 ng of DNA per sample were indexed and pooled for sequencing.

Next generation sequencing run. The pooled random subset library was applied to a next generation sequencing lane NovaSeq 6000 S4 flow cell to obtain ˜5× coverage (Illumina Inc., San Diego, CA, USA); the 5× coverage constitutes sequencing a random subset of each random set of cell-free DNA molecules.

Data processing. Raw sequencing reads were demultiplexed and subject-specific FASTQ files were analyzed. FASTQ files were aligned on the human genome (GRCh37, version hs37d5 including decoys). The subsequent processing pipeline consisted of trimming adapters and methylation bias, screening for contaminating genomes, aligning to the reference genome, removing PCR duplicates, calculating coverage, calculating insert size, extracting CpG methylation, generating a genome-wide cytosine report (CpG count matrix), as well as examining quality control metrics (see Laufer et al).

### Example 3

Disclosed is the process for sequencing by repetitive transient hybridization as described in U.S. patent application Ser. No. 16/425,929 that can be used for sequencing or molecular identification of cell-free DNA molecules and for detecting modifications.

Isolation of a random set of cellfree DNA molecules. 10 ml blood sample was collected from subject in a Streck-BCT tube (Streck, Omaha, NE). Plasma was isolated from sample by centrifuged at 3000×g for 15 min at 4° C.). cfDNA was extracted from 5 ml of plasma using the QIAamp circulating nucleic acid kit ((Qiagen, Valencia, CA). Total DNA was quantified using a Qubit dsDNAHS Assay kit (Life Technologies, Grand Island, NY, USA).

Preparation of a random set of cellfree DNA molecules. End-modification of the sample cfDNA was carried out by incubating the DNA with terminal transferase (TdT) and ddATP biotin; samples DNA was purified using a commercial DNA purification kit (Genejet).

Pre-washing flow cell and loading cell free DNA molecules and making single-stranded. A flow cell with a hydrogel and streptavidin coated glass surface (Schott AG, Mainz Germany) is washed with PBS Buffer. Biotin labelled sample DNA prepared above is added to the flow cell. After 4 min the flow cell is washed with 150 ul of PBS buffer. The flow cell is washed 5 times with freshly prepared 0.5M NaOH, including one two-minute incubation in NaOH at room temperature, before washing with buffer (Tris, MgCl2, EDTA, Tween 20, Water) 4 times.

Sequencing run. The flow cell is loaded on to a super-resolution nanoimager (Oxford nanoimaging) connected to a fluid delivery auto-sampler. The flow cell is primed with imaging buffer ((Tris, MgCl2, EDTA, Tween 20, Water, Oxygen scavenger system, e.g. Pyranose Oxidase, COT, Trolox) and a cycles of the following two steps are performed: 1. incubation with one or more fluorescently labelled LNA oligos in imaging buffer from a repertoire of 1024 5mers and simultaneous imaging. 2. Flushing out spent fluorescent oligos. At each step different one or more oligos are added. Imaging is performed using an evanescent field for illumination and a CMOS sensor for detection. Fluorophores are selected from Cy3 and atto 647N.

Dataprocessing. After sufficient cycles for delivery of the required set of fluorescently labelled oligos, each sub-set of oligos that find a sequence match in each of the immobilized sample DNA are compared in silico to a reference genome to map the location of each DNA molecule in the genome; this defines an identity for each DNA molecule. The kinetics of binding of the oligos along the immobilized molecules is used to determine the methylation status of oligo binding sites containing CpGs in the identified sample DNA molecules.

### REFERENCES CITED AND ALTERNATIVE EMBODIMENTS

Zviran, et al., 2020, “Genome-wide cell-free DNA mutational integration enables ultra-sensitive cancer monitoring,” Nat Med 26, pp. 1114-1124.
Aravanis and Klausne, 2017, “Next-Generation Sequencing of Circulating Tumor DNA for Early Cancer Detection,” Cell 168(4): pp. 571-574.
Liu et al., 2020, “Sensitive and specific multi-cancer detection and localization using methylation signatures in cell-free DNA,” Ann. Oncol. 31, 745-759.
Razavi et al., 2019, “High-intensity sequencing reveals the sources of plasma circulating cell-free DNA variants,” Nat Med 25, pp. 1928-1937, https://doi.org/10.1038/s41591-019-0652-7.
Chen et al., 2020, “Non-invasive early detection of cancer four years before conventional diagnosis using a blood test.,” Nat. Commun. 11, p. 3475.
Song et al., 2017, “5-Hydroxymethylcytosine signatures in cell-free DNA provide information about tumor types and stages,” Cell Res 27, 1231-1242, https://doi.org/10.1038/cr.2017.106.
Guler et al., 2020, “Detection of early stage pancreatic cancer using 5-hydroxymethylcytosine signatures in circulating cell free DNA,” Nat Commun 11, 5270.
Dias and Torkamani, 2019, “Artificial intelligence in clinical and genomic diagnostics,” Genome Med 11, 70, https://doi.org/10.1186/s13073-019-0689-8.
Bergamaschi et al., 2020, “Pilot study demonstrating changes in DNA hydroxymethylation enable detection of multiple cancers in plasma cell-free DNA,” medRxiv 2020.01.22.20018382; doi: https://doi.org/10.1101/2020.01.22.20018382.
Jungmann et al., 2010, “Single-Molecule Kinetics and Super-Resolution Microscopy by Fluorescence Imaging of Transient Binding on DNA Origami,” Nano letters 10 (11), pp. 4756-4761.
Eid et al., 2009, “Real-Time DNA Sequencing from Single Polymerase Molecules” Science 323(5910), pp. 133-138.
Liu et al., 2020, “Accurate targeted long-read DNA methylation and hydroxymethylation sequencing with TAPS,” Genome Biol 21, 54.
Jain et al., 2016, “The Oxford Nanopore MinION: delivery of nanopore sequencing to the genomics community” Genome biology 17, article number 239, pp. 1-11.
Bicci et al., “Oxford Nanopore sequencing-based protocol to detect CpG methylation in human mitochondrial DNA,” bioRxiv 2021.02.20.432086; doi: https://doi.org/10.1101/2021.02.20.432086.
Wan et al., 2020, “ctDNA monitoring using patient-specific sequencing and integration of variant reads,” Sci Transl Med. 12(548).
Liu et al., 2019, “Bisulfite-free direct detection of 5-methylcytosine and 5-hydroxymethylcytosine at base resolution,” Nat Biotechnol 37, 424-429.
Laufer et al., 2020, “Low-pass whole genome bisulfite sequencing of neonatal dried blood spots identifies a role for RUNX1 in Down syndrome DNA methylation profiles,” Human Molecular Genetics 29(21), pp. 3465-3476.
Chan et al., 2013, PNAS, 110 (47) 18761-18768.

All references cited herein are incorporated herein by reference in their entirety and for all purposes to the same extent as if each individual publication or patent or patent application was specifically and individually indicated to be incorporated by reference in its entirety for all purposes.

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope unless otherwise claimed. No language in the specification should be construed as indicating any non-claimed element as essential to the practice of the invention.

It will also be understood that, although the terms first, second, etc. is used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description and the appended claims, the singular forms “a”, “an” and “the” are intended to include the plural forms as well, unless the context clearly indicates otherwise. It will also be understood that the term “and/of” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is construed to mean “when” or “upon” or “in response to determining” or “in response to detecting,” depending on the context. Similarly, the phrase “if it is determined” or “if [a stated condition or event] is detected” is construed to mean “upon determining” or “in response to determining” or “upon detecting (the stated condition or event)” or “in response to detecting (the stated condition or event),” depending on the context.

The citation and incorporation of patent documents herein is done for convenience only and does not reflect any view of the validity, patentability, and/or enforceability of such patent documents.

The present invention can be implemented as a computer program product that comprises a computer program mechanism embedded in a non-transitory computer readable storage medium. For instance, the computer program product could contain the program modules shown in any combination of FIG. 1A. These program modules can be stored on a CD-ROM, DVD, magnetic disk storage product, USB key, or any other non-transitory computer readable data or program storage product.

The embodiments within the specification provide an illustration of embodiments of the invention and should not be construed to limit the scope of the invention. The skilled artisan will recognize that many other aspects and embodiments are encompassed by the methods of this invention. The embodiments of the invention and technical details provided below can be varied by the skilled artisan and can be tested and systematically optimized without undue experimentation or re-invention.

The invention is most thoroughly understood in light of the teachings of the specification and the references cited within. Many modifications and variations can be made without departing from its spirit and scope, as will be apparent to those skilled in the art. The specific embodiments described herein are offered by way of example, only. The embodiments were chosen and described in order to best explain the principles and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed:

1. A method for determining whether a test subject has a particular phenotype, the method comprising:
obtaining a plurality of sequences, wherein each sequence represents a sequence of a nucleic acid molecule in a plurality of nucleic acid molecules in a biological sample from the test subject;
mapping each sequence in the plurality of sequences to a reference genome of the species of the test subject;
determining, for each respective site available for epigenetic modification in a plurality of sites available for epigenetic modification, an epigenetic state of the respective site in each respective sequence in the plurality of sequences having the respective site;
using each sequence in the plurality of sequences mapped to the reference genome and the epigenetic state of each site, in the plurality of sites, available for epigenetic modification in each sequence in the plurality of sequences to form a state of epigenetic modification of each respective subset of a plurality of subsets of sites available for epigenetic modification in the plurality of sites available for epigenetic modification, wherein the plurality of subsets of sites represents a random subset of a reference plurality of sites available for epigenetic modification associated with the phenotype, thereby obtaining a plurality of epigenetic subset states, wherein
each respective epigenetic subset state in the plurality of epigenetic subset states corresponds to a respective subset in the plurality of subsets, and
each respective subset in the plurality of subsets comprises three or more sites available for epigenetic modification, in the plurality of sites available for epigenetic modification, whose epigenetic state is associated with absence or presence of the phenotype; and
comparing the plurality of epigenetic subset states to a reference plurality of epigenetic subset states associated with the phenotype, thereby determining whether the subject has the phenotype.

2. The method of claim 1, wherein the phenotype is absence or presence of a particular disease.

3. The method of claim 2, wherein the disease is cancer.

4. The method of claim 1, wherein the phenotype is a stage of a disease.

5. The method of claim 1, wherein the phenotype is a prognosis for a disease.

6. The method of any one of claims 1-5, wherein each respective subset of sites available for epigenetic modification are present on individual nucleic acid molecules in the biological sample represented by the plurality of sequences.

7. The method of any one of claims 1-6, wherein the plurality of subsets of sites available for epigenetic modification collectively encompasses at least two, three, four, five, six, seven, eight, nine, or ten percent of the sites available for epigenetic modification in the genome of the species.

8. The method of any one of claims 1-7, wherein the species is human.

9. The method of any one of claims 1-7, wherein the species is mammalian species.

10. The method of any one of claims 1-7, wherein the species is a plant species.

11. The method of claim 1 wherein the comparing comprises inputting the plurality of epigenetic subset states into a trained model to obtain an indication of whether the subject has or does not have the phenotype as output of the trained model.

12. The method of claim 11, wherein the trained model comprises a linear model.

13. The method of claim 12, wherein the linear model is a random forest, a support vector machine, a convolutional neural network, or a linear regression model.

14. The method of claim 11, wherein the indication is a binary classification.

15. The method of claim 11, wherein the indication is a likelihood or probability.

16. The method of any one of claims 1-15, wherein the plurality of nucleic acid molecules consists of cell free DNA molecules.

17. The method of any one of claims 1-15, wherein the reference genome comprises at least 1×10⁶ bases or at least 20×10⁶ bases.

18. The method of any one of claims 1-17, wherein each sequence in the plurality of sequences uniquely represents a nucleic acid in the plurality of nucleic acids.

19. The method of any one of claims 1-18, wherein the plurality of sequences is obtained from the biological sample in a manner that is free of locus-targeted enrichment.

20. The method of any one of claims 1-19, wherein the three or more modifiable sites of a subset in the plurality of subsets are contiguous modifiable sites in the reference genome.

21. The method of any one of claims 1-19, wherein the three or more modifiable sites of a subset in the plurality of subsets are contiguous modifiable sites in the reference genome.

22. The method of any one of claims 1-19, wherein the three or more modifiable sites of a subset in the plurality of subsets are non-contiguous modifiable sites in the reference genome.

23. The method of any one of claims 1-19, wherein the three or more modifiable sites of each subset in the plurality of subsets are non-contiguous modifiable sites in the reference genome.

24. The method of any one of claims 1-19, wherein
the three or more sites available for epigenetic modification of a first portion of the plurality of subsets are contiguous sites available for epigenetic modification in the reference genome, and
the three or more sites available for epigenetic modification of a second portion of the plurality of subsets are non-contiguous sites available for epigenetic modification in the reference genome.

25. The method of any one of claims 1-24, wherein each subset in the plurality of subsets uniquely represents at least 30, 50,100,150, 200, 250, 300 nucleotides of the reference genome.

25. The method of any one of claims 1-24, wherein each subset in the plurality of subsets uniquely represents at least 30, 50,100,150, 200, 250, 300 nucleotides of the reference genome.

26. The method of any one of claims 1-25, wherein the epigenetic state of the respective site in each respective sequence in the plurality of sequences having the respective site comprises a methylation state, a hydroxymethylation state or a combination thereof.

27. The method of any one of claims 1-26, wherein each subset in at least a portion of the plurality of subsets represents a different haplotype block in a plurality of haplotype blocks.

28. The method of any one of claims 1-27, wherein
each sequence in the plurality of sequences represents a different nucleic acid molecule in the plurality of molecules in the nucleic acid sample,
the plurality of sequences collectively comprises at least 4 genome equivalents of nucleic acids for the species or at least 40 genome equivalents of nucleic acids for the species.

29. The method of any one of claims 1-28, wherein the plurality of nucleic acid molecules are cell-free DNA molecules and the biological sample comprises blood, plasma, urine, stool, saliva, sputum, a throat swab, a nose swab, a nasopharyngeal swab, milk, hair follicle, skin, seroma or serosanguineous fluid, cerebrospinal fluid, or breath from the subject.

30. The method of any one of claims 1-28, wherein the biological sample consists of between 1 blood droplet and 5 blood droplets.

31. The method of claim 1, wherein the comparing determines the phenotype or a degree or a nature of the phenotype by a global extent of differences in modification in the state of epigenetic modification of each respective subset of the plurality of subsets of sites available for epigenetic modification in the plurality of sites from a model for the phenotype.

32. The method of claim 31, wherein the plurality of subsets maps to genes, regulatory elements or pathways having sites, available for epigenetic modification, associated with the phenotype.

33. The method of claim 1, the method further comprising performing longitudinal tracking of respective biological samples from the test subject over time to determine a longitudinal signal for phenotype.

34. The method of claim 33, wherein the longitudinal signal for phenotype represents absence or presence of a disease over time, absence or presence of a residual disease over time, a progression of a disease, a recurrence of the disease, or a clearance of the disease.

35. The method of any one of claims 1-34, wherein the obtaining, mapping, determining, using, and comparing is performed for a second subject and wherein the plurality of subsets of sites obtained for the second subject represents a different random subset of the reference plurality of sites available for epigenetic modification than the random subset of the reference plurality of sites available for epigenetic modification used for the first test subject.

36. A method for determining the cell type of or presence or absence of a phenotype in, a single cell, the method comprising:
determining a state of modification of a subset of sites available for epigenetic modification across the genome to yield a matrix of state likelihoods per corresponding site in the genome;
comparing the matrix of state likelihoods per corresponding site in the genome determined for the current cell against a computer model of states per corresponding site in the genome that correspond to a specific cell phenotype; and
determining the phenotype state of the cell based on a threshold applied by the computer model.

37. A method for determining the presence or absence of, or the nature of, a particular disease or phenotype in a subject comprising:
determining a state of modification (e.g., methylation) of a random subset of single or multiple-linked, modifiable nucleotides (e.g., CpG sites) across the genome;
selecting the nucleotides in silico according to the extent to which they are modified in populations with and without the disease or phenotype;
of the selected nucleotides, quantitatively determining in silico a proportion whose state of modification has diverged from a baseline according to a predetermined threshold to determine the presence or absence of the disease or phenotype;
wherein the composition of the random subset of loci across the genome is different from one subject to another.

38. A method according to claim 37, wherein the nucleotides are present on cell free nucleic acids.

39. A method of detecting a molecular signature for cancer comprising:
isolating a substantially random subset of molecules from a set of molecules in a nucleic acid sample inside a device;
determining an identity of individual molecules within the subset of molecules by obtaining sequence information from each individual molecule using a sequencing or sequence detection method inside the device and using the sequence information to map the molecule in silico to a location in the genome using one or more programmable computer processors and computer memory;
determining the methylation status of each of the molecules mapped to a location the genome in ii using a method for detecting presence or absence of, the extent of, or the pattern of methylation of modifiable nucleotides on individual molecules inside the device, and storing the resulting methylation information for each molecule in computer memory;
executing a computer program to filter out (eliminate from further consideration), all the modifiable nucleotides in the sequence of individual molecules in computer memory, which do not fulfill a predefined criteria and storing the resulting processed data of individual molecules in computer memory;
aggregating data on the processed methylation status of the individual molecules within the subset of molecules in computer memory; and
using a computer processor programmed to calculate/compute the proportion of molecules, in the aggregated data in computer memory whose methylation status has diverged from a baseline according to a predetermined threshold, to determine if a molecular signature for cancer is present, and provide it as a computer output optionally showing a confidence score.

40. The method according to claim 37 or 39, wherein the predefined criteria is that the same sites on molecules containing the same sequence are, depending on site, methylated or demethylated in one or more cancer patients.

41. The method according to claim 37 or 39, wherein the predefined criteria for including a particular site in the aggregated data is that the site is hypomethylated in >70% of cancer patients and is not hypomethylated in >30% of healthy individuals.

42. The method according to claim 37 or 39, wherein the predefined criteria for including a particular site in the aggregated data is that the site is hypomethylated in >80% of cancer patients and is not hypomethylated in >40% of healthy individuals.

43. The method according to claim 37 or 39, wherein the predefined criteria for including a particular site in the aggregated data is that the site is hypomethylated in >60% of cancer patients and is not hypomethylated in >10% of healthy individuals.

44. The method according to claim 37 or 39, wherein the predetermined threshold is that >0.01%, 0.1%, 1%, >10%, >20%, >30% of molecules fulfill the criteria.

45. The method according to claim 37 or 39, wherein the composition of the subset of molecules is different from one individual/subject to another.

46. The method according to claim 37 or 39, the method further comprising determining when a signal for cancer is present, the stage of cancer, the type of cancer and providing a prognosis and a possible re-testing and/or treatment plan.

47. The method according to claim 37 or 39, wherein longitudinal tracking of samples from the same subject is used to determine a signal for disease, residual disease, the progression of the disease, the recurrence of the disease, the clearance of the disease.

48. The method according to any one of the preceding claims, wherein the method is carried out whether the aim is to detect a specific cancer type or any cancer type.

49. The method according to any one of claims 1-48, where the signature identifies changes in molecular pathways, enabling insights into molecular mechanisms and targets for drug intervention to be identified.

50. The method of any one of claims 1-49, wherein the obtaining further comprises using a random selection process to select a subset of sequences determined for the plurality of nucleic acid molecules to be the plurality of sequences, and wherein the plurality of subsets of sites represent the random subset of the reference plurality of sites available for epigenetic modification associated with the phenotype, at least in part, on the basis of the random selection process used to select the plurality of sequences.

51. The method of any one of claims 1-50, wherein the plurality of sites available for epigenetic modification comprises 1000 or more sites, 2000 or more sites, 3000 or more sites, 5000 or more sites, 10,000 or more sites, 100,000 or more sites or 1×10⁶ or more sites.

52. The method of any one of claims 1-51, wherein the plurality of subsets of sites comprises 250 or more subsets, 500 or more subsets, 1000 or more subsets, 2000 or more subsets, 3000 or more subsets, 5000 or more subsets, 10,000 or more subsets, 100,000 or more subsets or 1×10⁶ or more subsets.

53. The method of claim 52, wherein each subset in the plurality of subsets consists of a different three or more modifiable sites in the plurality of modifiable sites.

54. The method of any one of claims 1-53, wherein the plurality of subsets of sites available for epigenetic modification collectively encompasses at least one percent of the sites available for epigenetic modification in the genome of the species.

55. The method of claim 1 of any one of claims 1-35, wherein the comparing the plurality of epigenetic subset states to a reference plurality of epigenetic subset states associated with the phenotype, thereby determining whether the subject has the phenotype comprises determining that the extent of hypomethylation in the DNA of the subject is greater than the extent of hypometheylation of at least one subject without the phenotype.

56. The method of claim 36 wherein the extent of hypomethylation is persistently greater at 2 or more contiguous modifiable sites along statistically significant number of molecules in the sample.

57. A computer system comprising:
one or more processors;
memory; and
one or more programs, wherein the one or more programs are stored in the memory and are configured to be executed by the one or more processors, the one or more programs for determining whether a test subject has a phenotype, the one or more programs including instructions for:
obtaining a plurality of sequences in electronic form, wherein each sequence represents a sequence of a nucleic acid molecule in a plurality of nucleic acid molecules in a biological sample from the test subject;
mapping each sequence in the plurality of sequences to a reference genome of the species of the test subject;
determining, for each respective site available for epigenetic modification in a plurality of sites available for epigenetic modification, an epigenetic state of the respective site in each respective sequence in the plurality of sequences having the respective site;
using each sequence in the plurality of sequences mapped to the reference genome and the epigenetic state of each site, in the plurality of sites, available for epigenetic modification in each sequence in the plurality of sequences to form a state of epigenetic modification of each respective subset of a plurality of subsets of sites available for epigenetic modification in the plurality of sites available for epigenetic modification, wherein the plurality of subsets of sites represents a random subset of a reference plurality of sites available for epigenetic modification associated with the phenotype, thereby obtaining a plurality of epigenetic subset states, wherein
each respective epigenetic subset state in the plurality of epigenetic subset states corresponds to a respective subset in the plurality of subsets, and
each respective subset in the plurality of subsets comprises three or more sites available for epigenetic modification, in the plurality of sites available for epigenetic modification, whose epigenetic state is associated with absence or presence of the phenotype; and
comparing the plurality of epigenetic subset states to a reference plurality of epigenetic subset states associated with the phenotype, thereby determining whether the subject has the phenotype.

58. A computer readable storage medium storing one or more programs, the one or more programs comprising instructions, which when executed by an electronic device with one or more processors and a memory cause the electronic device to determine whether a test subject has a phenotype by a method comprising:
obtaining a plurality of sequences in electronic form, wherein each sequence represents a sequence of a nucleic acid molecule in a plurality of nucleic acid molecules in a biological sample from the test subject;
mapping each sequence in the plurality of sequences to a reference genome of the species of the test subject;
determining, for each respective site available for epigenetic modification in a plurality of sites available for epigenetic modification, an epigenetic state of the respective site in each respective sequence in the plurality of sequences having the respective site;
using each sequence in the plurality of sequences mapped to the reference genome and the epigenetic state of each site, in the plurality of sites, available for epigenetic modification in each sequence in the plurality of sequences to form a state of epigenetic modification of each respective subset of a plurality of subsets of sites available for epigenetic modification in the plurality of sites available for epigenetic modification, wherein the plurality of subsets of sites represents a random subset of a reference plurality of sites available for epigenetic modification associated with the phenotype, thereby obtaining a plurality of epigenetic subset states, wherein
each respective epigenetic subset state in the plurality of epigenetic subset states corresponds to a respective subset in the plurality of subsets, and
each respective subset in the plurality of subsets comprises three or more sites available for epigenetic modification, in the plurality of sites available for epigenetic modification, whose epigenetic state is associated with absence or presence of the phenotype; and
comparing the plurality of epigenetic subset states to a reference plurality of epigenetic subset states associated with the phenotype, thereby determining whether the subject has the phenotype.
